SEQUENCE LISTING (66 sequences)

SEQ ID NO: 1 - protein, 9 aa, Artificial Sequence
  Description of Artificial Sequence Synthetic peptide
YLEPGPVTA

SEQ ID NO: 2 - protein, 10 aa, Artificial Sequence
  Description of Artificial Sequence Synthetic peptide
DYMDGTMSQV

SEQ ID NO: 3 - protein, 9 aa, Artificial Sequence
  Description of Artificial Sequence Synthetic peptide
ALLAVGATK

SEQ ID NO: 4 - protein, 9 aa, Artificial Sequence
  Description of Artificial Sequence Synthetic peptide
KCDICTDEY

SEQ ID NO: 5 - protein, 9 aa, Artificial Sequence
  Description of Artificial Sequence Synthetic peptide
RLSNRLLLR

SEQ ID NO: 6 - protein, 9 aa, Artificial Sequence
  Description of Artificial Sequence Synthetic peptide
SQNFPGSQK

SEQ ID NO: 7 - protein, 9 aa, Artificial Sequence
  Description of Artificial Sequence Synthetic peptide
KTWGQYWQV

SEQ ID NO: 8 - protein, 9 aa, Artificial Sequence
  Description of Artificial Sequence Synthetic peptide
  MOD_RES (2)..(2): Thr or Met
IXDQVPFSV

SEQ ID NO: 9 - protein, 10 aa, Artificial Sequence
  Description of Artificial Sequence Synthetic peptide
VLYRYGSFSV

SEQ ID NO: 10 - protein, 9 aa, Artificial Sequence
  Description of Artificial Sequence Synthetic peptide
AAGIGILTV

SEQ ID NO: 11 - protein, 9 aa, Artificial Sequence
  Description of Artificial Sequence Synthetic peptide
ALNFPGSQK

SEQ ID NO: 12 - protein, 9 aa, Artificial Sequence
  Description of Artificial Sequence Synthetic peptide
LIYRRRLMK

SEQ ID NO: 13 - protein, 9 aa, Artificial Sequence
  Description of Artificial Sequence Synthetic peptide
AAQERRVPR

SEQ ID NO: 14 - protein, 10 aa, Artificial Sequence
  Description of Artificial Sequence Synthetic peptide
ASGPGGGAPR

SEQ ID NO: 15 - protein, 9 aa, Artificial Sequence
  Description of Artificial Sequence Synthetic peptide
LLGPGRPYR

SEQ ID NO: 16 - protein, 13 aa, Artificial Sequence
  Description of Artificial Sequence Synthetic peptide
SDAEKSDICTDEY

SEQ ID NO: 17 - protein, 11 aa, Artificial Sequence
  Description of Artificial Sequence Synthetic peptide
SSDYVIPIGTY

SEQ ID NO: 18 - protein, 9 aa, Artificial Sequence
  Description of Artificial Sequence Synthetic peptide
EADPTGHSY

SEQ ID NO: 19 - protein, 9 aa, Artificial Sequence
  Description of Artificial Sequence Synthetic peptide
EVDPIGHLY

SEQ ID NO: 20 - protein, 10 aa, Artificial Sequence
  Description of Artificial Sequence Synthetic peptide
DYMDGTMSQV

SEQ ID NO: 21 - protein, 9 aa, Artificial Sequence
  Description of Artificial Sequence Synthetic peptide
IMDQVPFSV

SEQ ID NO: 22 - protein, 9 aa, Artificial Sequence
  Description of Artificial Sequence Synthetic peptide
YLEPGPVTA

SEQ ID NO: 23 - protein, 9 aa, Artificial Sequence
  Description of Artificial Sequence Synthetic peptide
GLYDGMEHL

SEQ ID NO: 24 - protein, 9 aa, Artificial Sequence
  Description of Artificial Sequence Synthetic peptide
LIYRRRLMK

SEQ ID NO: 25 - protein, 10 aa, Artificial Sequence
  Description of Artificial Sequence Synthetic peptide
ASGPGGGAPR

SEQ ID NO: 26 - protein, 16 aa, Artificial Sequence
  Description of Artificial Sequence Synthetic peptide
AQNILLSNAPLGPQFP

SEQ ID NO: 27 - protein, 21 aa, Artificial Sequence [sequence omitted]

SEQ ID NO: 28 - protein, 23 aa, Artificial Sequence
  Description of Artificial Sequence Synthetic peptide
RNGYRALMDKSLHVGTQCALTRR

SEQ ID NO: 29 - protein, 15 aa, Artificial Sequence
  Description of Artificial Sequence Synthetic peptide
TSYVKVLHHMVKISG

SEQ ID NO: 30 - protein, 14 aa, Artificial Sequence
  Description of Artificial Sequence Synthetic peptide
LLKYRAREPVTKAE

SEQ ID NO: 31 - protein, 16 aa, Artificial Sequence
  Description of Artificial Sequence Synthetic peptide
WNRQLYPEWTEAQRLD

SEQ ID NO: 32 - protein, 9 aa, Artificial Sequence
  Description of Artificial Sequence Synthetic peptide
KIFGSLAFL

SEQ ID NO: 33 - protein, 16 aa, Artificial Sequence
  Description of Artificial Sequence Synthetic peptide
AQYIKANSKFIGITEL

SEQ ID NO: 34 - protein, 9 aa, Artificial Sequence
  Description of Artificial Sequence Synthetic peptide
YLSGADLNL

SEQ ID NO: 35 - protein, 9 aa, Artificial Sequence
  Description of Artificial Sequence Synthetic peptide
VLRENTSPK

SEQ ID NO: 36 - protein, 9 aa, Artificial Sequence
  Description of Artificial Sequence Synthetic peptide
EADPTGHSY

SEQ ID NO: 37 - protein, 9 aa, Artificial Sequence
  Description of Artificial Sequence Synthetic peptide
EIWTHSYKV

SEQ ID NO: 38 - protein, 9 aa, Artificial Sequence
  Description of Artificial Sequence Synthetic peptide
SLFRAVITK

SEQ ID NO: 39 - protein, 9 aa, Artificial Sequence
  Description of Artificial Sequence Synthetic peptide
EVDPIGHLY

SEQ ID NO: 40 - protein, 9 aa, Artificial Sequence
  Description of Artificial Sequence Synthetic peptide
GLYDGMEHL

SEQ ID NO: 41 - protein, 9 aa, Artificial Sequence
  Description of Artificial Sequence Synthetic peptide
HLFGYSWYK

SEQ ID NO: 42 - protein, 10 aa, Artificial Sequence
  Description of Artificial Sequence Synthetic peptide
ASGPGGGAPR

SEQ ID NO: 43 - protein, 9 aa, Artificial Sequence
  Description of Artificial Sequence Synthetic peptide
APPAYEKLS

SEQ ID NO: 44 - protein, 9 aa, Artificial Sequence
  Description of Artificial Sequence Synthetic peptide
PPAYEKLSA

SEQ ID NO: 45 - protein, 9 aa, Artificial Sequence
  Description of Artificial Sequence Synthetic peptide
PAYEKLSAE

SEQ ID NO: 46 - protein, 20 aa, Artificial Sequence
  Description of Artificial Sequence Synthetic peptide
VPNAPPAYEKLSAEQSPPPY

SEQ ID NO: 47 - protein, 12 aa, Artificial Sequence
  Description of Artificial Sequence Synthetic peptide
PNAPPAYEKLSA

SEQ ID NO: 48 - protein, 12 aa, Artificial Sequence
  Description of Artificial Sequence Synthetic peptide
NAPPAYEKLSAE

SEQ ID NO: 49 - protein, 12 aa, Artificial Sequence
  Description of Artificial Sequence Synthetic peptide
APPAYEKLSAEQ

SEQ ID NO: 50 - protein, 15 aa, Artificial Sequence
  Description of Artificial Sequence Synthetic peptide
APPAYEKLSAEQSPP

SEQ ID NO: 51 - protein, 16 aa, Artificial Sequence
  Description of Artificial Sequence Synthetic peptide
APPAYEKLSAEQSPPP

SEQ ID NO: 52 - protein, 16 aa, Artificial Sequence
  Description of Artificial Sequence Synthetic peptide
APPAYEKLSAEQSPPP

SEQ ID NO: 53 - protein, 12 aa, Artificial Sequence
  Description of Artificial Sequence Synthetic peptide
PPAYEKLSAEQS

SEQ ID NO: 54 - protein, 12 aa, Artificial Sequence
  Description of Artificial Sequence Synthetic peptide
PAYEKLSAEQSP

SEQ ID NO: 55 - protein, 12 aa, Artificial Sequence
  Description of Artificial Sequence Synthetic peptide
AYEKLSAEQSPP

SEQ ID NO: 56 - protein, 12 aa, Artificial Sequence
  Description of Artificial Sequence Synthetic peptide
YEKLSAEQSPPP

SEQ ID NO: 57 - protein, 12 aa, Artificial Sequence
  Description of Artificial Sequence Synthetic peptide
APPAYEKLSAEQ

SEQ ID NO: 58 - protein, 15 aa, Artificial Sequence
  Description of Artificial Sequence Synthetic peptide
APPAYEKLSAEQSPP

SEQ ID NO: 59 - protein, 16 aa, Artificial Sequence
  Description of Artificial Sequence Synthetic peptide
APPAYEKLSAEQSPPP

SEQ ID NO: 60 - DNA, 69 bp, Artificial Sequence
  Description of Artificial Sequence Synthetic oligonucleotide
cagttctgcgcagcttcccgaggctcgggaagctgcgcagaactggtcgggaagctgcgcagaactggg

SEQ ID NO: 61 - DNA, 69 bp, Artificial Sequence
  Description of Artificial Sequence Synthetic oligonucleotide
cacatgtgtaatacatatctggggcacatgtgtaatacatatctggtacacatgtgcacacacagaagg

SEQ ID NO: 62 - DNA, 923 bp, Artificial Sequence
  Description of Artificial Sequence Synthetic polynucleotide
  misc_feature (711)..(780): This region may or may not be present in its entirety
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtt

SEQ ID NO: 63 - protein, 15 aa, Artificial Sequence
  Description of Artificial Sequence Synthetic peptide
QYIKANSKFIGITEL

SEQ ID NO: 64 - protein, 42 aa, Unknown [sequence omitted]

SEQ ID NO: 65 - protein, 16 aa, Artificial Sequence
  Description of Artificial Sequence Synthetic peptide
AQYIKANSKFIGITEL

SEQ ID NO: 66 - protein, 12 aa, Unknown
  Description of Unknown PADRE sequence
  MOD_RES (1)..(1): D-alanine
  MOD_RES (3)..(3): L-cyclohexylalanine
  MOD_RES (3)..(3): D-alanine
AKAVAAWTLKAA

(19) United States
(12) Patent Application Publication
(10) Pub. No.: US 2019/0381157 A1
(43) Pub. Date: Dec. 19, 2019

(54) METHODS OF IMMUNE MODULATION AGAINST FOREIGN AND/OR AUTO ANTIGENS

(71) Applicant: ZEQUN TANG, San Ramon, CA (US)

(72) Inventor: Zequn TANG, SAN RAMON, CA (US)

(21) Appl. No.: 16/479,902

(22) PCT Filed: Jan. 29, 2018
(86) PCT No.: PCT/US18/15771

§ 371 Date: Jul. 22, 2019

Related U.S. Application Data
(60) Provisional application No. 62/451,759, filed on Jan. 29, 2017.

(51) Int. Cl. A61K 39/00; A61K 39/08; A61K 39/104; A61K 39/39

(52) U.S. Cl. CPC A61K 39/001106; A61K 39/08; A61K 39/0008; A61K 2039/53; A61K 39/104; A61K 39/39; A61K 2039/505; A61K 39/0007

(57) ABSTRACT

The present invention is directed to compositions and methods of treating proliferative disorders, such as cancer, using a composition comprising an immune checkpoint antagonist and an antigen. It has been found that the composition elicits an immune response allowing an otherwise suppressed immune system to activate in order for T cells to attack cancer cells. Compositions may be presented in a vector comprising nucleic acids encoding the antagonist and antigen, and may include gene editing systems, such as CRISPR-Cas9 system.

### CROSS-REFERENCE TO RELATED APPLICATION

This application claims the benefit of priority under 35 U.S.C. 35 USC § 119(e) to U.S. Application Ser. No. 62/451,759, filed Jan. 29, 2017, The disclosure of the prior applications is considered part of and is incorporated by reference in its entirety in the disclosure of this application.

### FIELD OF THE INVENTION

This invention related to the enhancement of immune responses to foreign or auto antigens, and can be used to improve the efficacy of therapeutic and/or prophylactic vaccines for various diseases, including cancers, infectious diseases, neurological degenerative diseases, autoimmune diseases, transplant rejection, and animal autoimmune disease model generation. In particular, the development of a novel tumor vaccine platform combining antigen expression with modulation of immune checkpoint-associated proteins within antigen presenting cells is described herein to improve the efficacy and safety of immune therapy.

### BACKGROUND OF THE INVENTION

Immune checkpoints have been successfully modulated to enhance immune response or to release immune suppression to treat various tumors. Certain cancer cells and ligands can bind to receptors on activated T cells to turn these cells off Immune checkpoint inhibitors act by preventing T cells from being turned off, thereby allowing activated T cells to attack infected or cancerous tumor cells and thus increasing a patient's own immune response.

Currently, immune checkpoint inhibitors, such as antibodies against PD-1/PDL1 and CTLA-4, represent a revolution in the treatment of cancer. However, many improvements are still urgently needed. The checkpoint inhibitors elicited durable responses only in a fraction of patients (20%-30%) of various solid tumor types, and due to their unselective/general de-repression of immune response, they also induce a plethora of autoimmune-related adverse events, which mainly include gastrointestinal, dermatological, hepatic and endocrinological toxicities. (Gelao, 2014.) While the exact mechanism causing the low response rates is not known, one of the potential mechanisms may be the lack of tumor antigen reactive clones in a patient's T cell repertoire prior to immune checkpoint inhibitor therapy. Several lines of evidence suggest that checkpoint proteins, besides acting on the T effector cells, are also expressed by the antigen presenting cells (APCs), and may play an important role in early antigen priming and immune response. (Spoonas, 2015; Raya et al., 2015; Weiskopf, 2015) Animal data demonstrated that targeting the PD-1/PD-L1 pathway with blocking monoclonal antibodies may enhance the therapeutic protection induced by a tumor vaccine (Remy-Ziller, 2017), CTLA-4 blockade can expand the peripheral T-cell receptor repertoire (Robert 2014). PD-L1 expressed by immune cells also plays an important role in CD8⁺ T cell priming, contraction, and differentiation into memory populations (Johnson, 2017) or memory T cell expansion. A clinical study showed that vaccine-primed patients may be better candidates than vaccine-naïve patients for immune checkpoint and other immunomodulatory therapies (Lutz, 2014). All these data support the feasibility of exploring further the combination of tumor vaccines with immune checkpoint modulation.

The exact role of immune checkpoint in antigen priming is not clear. It is possible that similar molecule mechanisims may be used for antigen priming in APCs and tumor killing in T effector cell. T cells reactive to antigens may be of low abundance in vaccine naive patients, even in tumors with high mutation load such as melanoma (Ott, Nature 2017), pre-priming and amplification of neoantigen specific immune effector cell clones and/or memory T cell expansion will be of great potential. It is speculated that B7-CD28 interaction serves differential roles in different immune responses or at different stages of a response: CD28:B7 interaction is important in activating naïve T cells, CTLA-4:B7 inhibit the T cell initial activation in secondary lymphoid organs, while PD1:PDL1 inhibits the activation of effector cells. (Abbas, 2018.) The exact impact of immune checkpoint on immune priming still needs to be further assessed.

The current invention involves expression of neoantigens as well as using molecular techniques to modulate (e.g., downregulating) the activity of checkpoint and ligands (e.g, PDL-1/2, B7H4, B7-1/2, etc.) within antigen presenting cells (APCs), to elicit effective immune recognition/priming for the neoantigen epitopes, expand the abundance of neoantigen specific T memory cell clones and increase the ratio of anti-neoantigen T cell clones in entire T cell repertoires, either by expanding the memory T cells for the specific antigen already in the patient or by priming naïve T cells against the neoantigens. This approach could be used alone, used prior to, or concurrently or after with the checkpoint inhibitor(s). In this manner, instead of generalized immune de-repression by current immune checkpoint inhibitors therapy, the modified therapy will first selectively expand the neoantigen reactive T cell clones and increase the likelihood of successful subsequent immune checkpoint therapy. Since epitope dominance and affinity maturation in T cell response are the result of competitive interactions between antigen-bearing APC and T cell subsets, the current invention may also reduce the autoimmune side effects by reducing the relative ratio of self-reacting T cell clones in T-cell pool. Lytic vector(s), nanoparticle coated antigenic epitopes, and immumodulators may also be used. This configuration can be used along with or without at least one other immunopotentiating cytokines/proteins (e.g. IFN-alpha, IFN-gamma, IL-2, IL-12, IL-15, IL-18, IL-21, GM-CSF, Flt-3 ligand etc.), within antigen presenting cells (APCs), to induce immune activation for the tumor epitopes.

Another configuration of the current invention involves expression of autoantigens as well as using molecular techniques to modulate (e.g., upregulating) the activity of checkpoint and ligands (e.g, PDL-1/2, B7H4, B7-1/2, etc.), along with or without at least one other immunosuppressive cytokines/proteins (e.g. IL-1Ra, IL-4, IL-10, IL-11, IL-13, TGF-b, IL-33, IL-35, IL-37, etc.), within antigen presenting cells (APCs), to induce immune tolerance for the autoantigen epitopes.

### SUMMARY OF THE INVENTION

The present invention is directed to a novel tumor vaccine platform, which combines antigen expression with modulation of immune checkpoint within antigen presenting cells (APCs), to improve the efficacy and safety of immune therapy. In one embodiment, the product may be a vaccine delivery vector or nanoparticles expressing a tumor specific antigen and immune checkpoint modulating elements. In one embodiment, the vector may comprise nucleic acids encoding, for example, Cas9 protein, and nucleic acids encoding for PDL1 and/or CTLA4 knock-out or knock-down.

The present invention is directed to a combination of at least one immune checkpoint antagonist and at least one antigen. Preferably, the composition comprises at least one nucleic acid encoding the immune checkpoint antagonist, which may be a polypeptide or protein that binds or otherwise interferes with the function or expression of an immune checkpoint-associated protein, or its RNA. In another embodiment, the polypeptide or protein is an antibody, such as a monoclonal antibody, humanized antibody, fully human antibody, a fusion protein, or a combination thereof. Alternatively, the polypeptide may be a fragment of such antibodies or protein that bind to the immune checkpoint-associated protein. It is further possible that the immune checkpoint antagonist is a small molecule that interferes with, inhibits, or otherwise disrupts the function of an immune checkpoint-associated protein or the expression thereof.

In one aspect of the invention, the immune checkpoint-associated protein is a ligand, such as PD-L1, PD-L2, CD80, CD86, ICOS Ligand, B7-H3, B7-H4, 4-1BBL, HVEM, OX40L,CD70, CD4OL, Galectin-9, Adenosine, GITRL, IDO, TDO, CEACAM1, VISTA, CD47, CD155, CD48, HHLA2, BTN2A1, BTN2A2, BTN3A1, BTNL3 and BTNL9.

In another aspect of the invention, the immune checkpoint-associated protein is a soluble receptor selected from the group consisting of PD-1, CTLA-4, CD28, ICOS, 4-1BB, BTLA, CD160, LIGHT, LAG3, OX40, CD27, CD40, TIM-3, Adenosine A2a receptor, GITR, CEACAM1, SIPR-alpha, DNAM-1, TIGIT, CD96, 2B4, TMIGD2, and DC-SIGN.

In a further embodiment, the immune checkpoint antagonist is preferably a nucleic acid for impairing or eliminating immune checkpoint associated protein expression or function, which may be in the form of an siRNA, microRNA, double stranded RNA, dicer substrate RNA, ribozyme, an aptamer, ALENs (transcription activator-like effector nuclease), or ZFNS (zinc finger nuclease). The immune checkpoint associated protein is selected from the group consisting of PD-L1, PD-L2, CD80, CD86, ICOS Ligand, B7-H3, B7-H4, 4-1BBL, HVEM, OX40L,CD70, CD40L, Galectin-9, Adenosine, GITRL, IDO, TDO, CEACAM1, VISTA, CD47, CD155, CD48, HHLA2, BTN2A1, BTN2A2, BTN3A1, BTNL3, BTNL9, PD-1, CTLA-4, CD28, ICOS, 4-1BB, BTLA, CD160, LIGHT, LAG3, OX40, CD27, CD40, TIM-3, Adenosine A2a receptor, GITR, CEACAM1, SIPR-alpha, DNAM-1, TIGIT, CD96, 2B4, TMIGD2, and DC-SIGN. Suitable antagonists include, but are not limited to CTLA-4 antagonist, vermurafenib, ipilimumab, dacarbazine, IL-2, temozolomide, imatinib, gefitinib, erlotinib, sunitinib, tyrphostins, a PD-1 agonist/antagonist and telatinib. In one embodiment, the antagonist is an antibody, such as, a CTLA-4 antagonist, avelumab, pembrolizumab, ipilimumab, durvalumab, atezolizumab, nivolumab, and a PD-1 agonist/antagonist. In other embodiments of the invention, the nucleic acid is codon optimized to yield a more stable mRNA than one encoding the same protein, which has not been so optimized.

In another embodiment, the immune checkpoint antagonist comprises at least one set of CRISPR-CAS9-encoding nucleic acid sequences capable of impairing or eliminating expression of an immune checkpoint associated protein selected from the group consisting of PD-L1, PD-L2, CD80, CD86, ICOS Ligand, B7-H3, B7-H4, 4-1BBL, HVEM, MHC-Class I, MHC Class II, OX40L,CD70, CD40L, Galectin-9, Adenosine, GITRL, IDO, TDO, CEACAM1, VISTA, CD47, CD155, CD48, HHLA2, BTN2A1, BTN2A2, BTN3A1, BTNL3, BTNL9, PD-1, CTLA-4, CD28, ICOS, 4-1BB, BTLA, CD160, LIGHT, LAG3, OX40, CD27, CD40, TIM-3, Adenosine A2a receptor, GITR, CEACAM1, SIPR-alpha, DNAM-1, TIGIT, CD96, 2B4, TMIGD2, and DC-SIGN. In this embodiment, the immune checkpoint antagonist may further comprise at least one guide RNA that targets a gene encoding an immune checkpoint associated protein.

In accordance with the present invention, the antigen may be a self-antigen, foreign antigen, or neoantigen. It may be a tumor-associated antigen, such as a cancer-testes associated antigen. In an embodiment of the invention, the antigen is a peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, -Catenin, CDK4, Mum-1, p16, TAGE, PSMA, PSCA, CT7, telomerase, 43-9F, 5T4, 791Tgp72, alpha-fetoprotein, beta-HCG, BCA225, BTAA, CA 125, CA 15-3 (CA 27.29/BCAA), CA 195, CA 242, CA-50, CAM43, CD68/KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

In accordance with one embodiment, the compositions of the present invention include an antigen present in the form of a nucleotide sequence encoding the antigen. Accordingly, the nucleotide sequence encoding the antigen is present on a vector that is delivered to a patient along with the delivery of the immune checkpoint antagonist.

In another embodiment, the antigen may comprise at least one neoantigen polypeptide that is mutated relative to wild type. This mutant polypeptide may bind to an MHC-Class I or MHC-Class II molecule. Moreover, the mutation relative to wild type in the neoantigen polypeptide is optionally identified by sequencing a portion of a tumor cell's genome and comparing that tumor sequence to a corresponding portion of a healthy donor cell's genome. In one embodiment, the sequence is done by next generation sequencing (NGS).

A neoantigen within the scope of the invention may be present across certain patient populations and tumor types. Optionally, it is encoded by an oncogene. In one embodiment, the antigen is recognized by a T Cell Receptor (TCR).

Compositions of the present invention may comprise at least one nucleic acid encoding the at least one immune checkpoint antagonist and at least one nucleic acid encoding the at least one antigen. In an embodiment of the invention, immune checkpoint antagonist and antigen are encoded by the same nucleic acid molecule. In another embodiment, the immune checkpoint antagonist and antigen are encoded by different nucleic acid molecules. The composition may comprise a single, two, or more plasmids or nucleic acid molecules.

The compositions of the present invention may further comprise an adjuvant. In an embodiment of the invention, the adjuvant is selected from the group consisting of montanide ISA-51, QS-21, tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), incomplete Freunds adjuvant, complete Freunds adjuvant, mineral gels, aluminum hydroxide (Alum), lysolecithin, pluronic polyols, polyanions, peptides, oil emulsions, dinitrophenol, and diphtheria toxin (DT).

The compositions of the invention are delivered to patients in need of therapeutic or preventive treatment. In one aspect of the invention, the composition may comprise cells that have been manipulated in vitro to insert the at least one immune checkpoint antagonist and the at least one antigen. Such cells may be autologous or heterologous to the patient. Preferably, the cells are autologous. In another embodiment, the cells are antigen presenting cells (APCs.)

The present invention is further directed to a method of treating a patient in need of the compositions of the present invention. For example, the patient may suffer from a disease, which is a proliferative disorder, such as cancer. The compositions of the presently disclosed subject matter can in some embodiments be used as a treatment for cancer, and more specifically for melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, ovarian cancer, and cervical cancer.

In an embodiment of the invention, the method of treating patients will result in at least or about a 10 to 20 percent reduction in cancer tumor size relative to standard treatment without the composition. In another embodiment, the patients are treated to prevent a proliferative disorder, such as cancer.

In another embodiment, the composition may comprise a vaccine delivery vector, e.g., Modified Vaccinia virus Ankara (MVA), adeno-associated virus (AAV), or lentiviral vectors, or nanoparticles expressing autoantigen and immune checkpoint proteins. In one embodiment, the vector may also encode at least one other immunosuppressive cytokines/proteins, such as IL-1Ra, IL-4, IL-10, IL-11, IL-13, TGF-b, IL-33, IL-35, IL-37, etc.

Autoantigens in the above embodiment may include, but are not limited to, histidine-tRNA ligase, ribonucleoproteins, snRNP core proteins, type I topoisomerase, histones, nucleoporin62, Sp100 nuclear antigen, nucleoporin 210Kda, actin, cyclic citrullinated peptide, thrombin, exosome complex proteins, nicotine acetylcholine receptor, muscle specific kinase, voltage-gated calcium channel, thyroid peroxidase, thyroglobulin, TSH receptor, Neuronal nuclear proteins, glutamate receptor, amphiphysin, glutamate decarboxylase, collapsing response mediator protein 5, N-methyl-D-aspartate receptor, aquaporin, and also include individual specific autoantibody (ISA) from antibody profiling technology described adequately in literature. It also includes donor antigens related to organ transplantation rejection.

The compositions of the presently disclosed subject matter can in some embodiments be used as a treatment for autoimmune diseases, and more specifically for systemic lupus erythematosus, Sjogren's syndrome, SLE, Mixed connective tissue disease, Primary billary cirrhosis, coeliac disease, rheumatoid arthritis, myasthenia Gravis, scleromyositis, Graves disease, Hashimoto's thyroiditis, paraneoplastic cerebellar degeneration, limbic encephalitis, encephalomyelitis, choreathetosis, subacute sensory neuronopathy, stiff person syndrome, diabetes mellitus type 1, optic neuropathy, chorea, and Devics syndrome (neuromyelitis optica).

The compositions of the presently disclosed subject matter can in some embodiments be used as a treatment for organ transplantation rejection. In this manner, the transplantation rejection-related antigen includes, but are not limited to, all the donor unmatched HLA types and non-HLA antigens. Donor unmatched HLA types may be identifies using publicly available computer algorithms that determine HLA matching at the epitope level (e.g., HLAMatchmaker), such as HLA AI (Eplet163RG), A1+A36 (44KM₂), A2 (66RKH), A2+A28 (142MT), A2+A69 (107W), A3 (161D), A9 (66GKH), A10 (149TAH), A11 (151AHA), A25+A32 (76ASI), A29 (62LQ), A30 (152RW), A30+A31(56R), A31+A33 (73ID), A68 (245VA), B5+B35 (193PV), B7 (177DK), B8+Cw7 (9D), B12 (167ES), B13 (144QL), B15 (163LW), B16 (158T), B17 (71SA), B18 (30G), B18+B35 (44RT), B27 (71KA), B40 (44RK), B44 (199V),B48 (245TA), Bw4 (82LR), Bw6 (80ERN), Cwl (6K), Cw2 (211T), Cw3 (173K), Cw4 (17WR), DR51 (96EN₃), Cw5+8 (138K), Cw7 (193PL), DR1 (12LKF₂), DR1+10 (13FEL), DR1+51 (96EV), DR2 (142M₂), DR3 (74R), DR4 (96Y2), DR3+6 (31YYFH), DR7 (25Q₃), DR7+9 (78V2), DR8 (25YRF), DR8+12 (16Y), DR9 (13FEY), R10 (40YD₂), DR11 (57DE), DR12 (37L), DR13 (71DEA), DR14 (57AA), DR15 (71A), DR17 (26TYD), DR51 (96EN₃), DR52 (98Q), DR53 (48YQ6), DQ7+9 (56PPD), DQ8 (56PPA), DQ7 (45EV), DQ6 (125G), DQ5 (74SR₃), DQ4 (56L₂), DQ3 MICA,(55PP), DQ2 (45GE₃), and DQ1 (52PQ2). NonHLA antigens include, but are not limited to, VACM1, ICAM1, Vimentin, Cardiac Myosin, Collagen V, k-al tubulin, and angiotensin II receptor type I.

In another embodiment, the invention is directed to a method of generating an animal autoimmune diseases model comprising administering to an animal the composition of the present invention comprising an antigen. In this method, the animal's immune system is modulated against the antigen with immune checkpoint antagonist(s). In another embodiment, the antigen is an autoantigen.

### BRIEF DESCRIPTION OF THE DRAWINGS

A more complete understanding of the presently disclosed subject matter can be obtained by reference to the accompanying Figures, when considered in conjunction with the subsequent detailed description. The embodiments illustrated in the Figures are intended to be exemplary only, and should not be construed as limiting the presently disclosed subject matter to the illustrated embodiments.

FIG. 1 depicts alternative embodiments of vectors comprising antagonists and antigens within the scope of the invention, including CRISPR-Cas9 protein under control of the CMV promoter, a guide RNA under control a T7 promoter, and the beta-amyloid under control of the hEFla promoter. Alternatives including substitution of the beta-amyloid for Her2/Neu or MUC1 and/or WT1 and/or CEA.

FIG. 2 depicts alternative embodiments of vectors comprising CRISPR-Cas9 protein as expressed from CMV promoter, two guide RNAs as expressed from T7 promoter, and the Her2/Neu as expressed from h-EFla promoter. Alternatives shown include substituting beta-amyloid for truncated PDL1, Her2/Neu, or MUC1 and/or WT1 and/or CEA. In this configuration, two immune checkpoint associated proteins will be impaired or eliminated.

FIG. 3 depicts alternative embodiments of a vector comprising CRISPR-Cas9 protein as expressed from CMV promoter, guide RNA against PDL1 as expressed from T7 promoter, and the beta-amyloid as expressed from subgenomic promoter of an alphavirus replicon.

FIG. 4 depicts an embodiment of the invention in which a DNA vector is constructed in which the PDL1 and/or CTLA4 protein under the control of the CMV promoter, PDL1 protein is under control of the PGK promoter, and BRAF is expressed under the hEFla promoter and/or peptidyl arginine deiminase-4 protein is expressed under the SV40 promoter.

FIG. 5 depicts a construct comprising CTLA4 protein expressed under the CMV promoter, PDL1 antigen expressed under the PGK promoter, VACM1 expressed under the hEFla promoter and/or donor unmatched HLA DQ7+9 (56PPD) expressed under the SV40 promoter.

### DETAILED DESCRIPTION

The present invention is directed to a composition and methods comprising at least one immune checkpoint antagonist; and at least one antigen.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of +/−20%, in some embodiments +/−10%, in some embodiments +/−5%, in some embodiments +/−1%, in some embodiments +/−0.1%, in some embodiments +/−0.5%, and in some embodiments +/−0.01% from the specified amount, as such variations are appropriate to perform the disclosed methods.

As used herein, the term “and/or” when used in the context of a list of entities, refers to the entities being present singly or in any possible combination or sub combination. Thus, for example, the phrase “A, B, C, and/or D” includes A, B, C, and D individually, but also includes any and all combinations and subcombinations of A, B, C, and D.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino adds, The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino adds, etc.), as well as other modifications known in the att. Itis understood that, because the polypeptides of this invention are based upon an antibody, the polypeptides can occur as single chains or associated chains.

“Polynucleotide,” or “nucleic acid,” or “nucleotide sequence” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, A polynucleotide may comprise modified nucleotides, such as methylated nucleotides and their analogs. if present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“di thioate”), (O)NR₂ (“amidate”), P(O)R, P(O)OR′, CO or CH₂ (“formacetal”), in which each R or R′ is independently H or substituted or unsuhstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An epitope that “preferentially binds” or “specifically binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a specified epitope is an antibody that binds this epitope with greater affinity, avidity, more readily, and/or with greater duration than it binds to other non-specified epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “treatment” is an approach for obtaining beneficial or desired clinical results. For purposes of this invention, beneficial or desired clinical results include, but are not limited to, one or more of the following: improvement in any aspect of diminishing symptoms of a proliferative disorder, such as cancer, a neurodegenerative disorder, such as Alzheimer's, an autoimmune disorder, an infectious disease, or organ transplant rejection.

“Ameliorating” one or more symptoms of cancer means a lessening or improvement of one or more symptoms of cancer patients as compared to not administering an a composition of the present invention comprising an immune checkpoint antagonist in combination with an antigen. “Ameliorating” also includes shortening or reduction in duration of a symptom in general.

“Development” or “progression” of headache means initial manifestations and/or ensuing progression of the disorder. Development of headache can be detectable and assessed using standard clinical techniques as well known in the art. However, development also refers to progression that may be undetectable. For purpose of this invention, development or progression refers to the biological course of the symptoms. “Development” includes occurrence, recurrence, and onset. As used herein “onset” or “occurrence” of headache includes initial onset and/or recurrence.

An “individual” or a “subject” is a mammal, more preferably a human. Mammals also include, but are not limited to, farm animals, sport animals, pets, primates, horses, dogs, cats, mice and rats.

As used herein, “vector” means a construct, which is capable of delivering, and preferably expressing, one or more gene(s) or sequence(s) of interest in a host cell. Examples of vectors include, but are not limited to, viral vectors, e.g., MVA, AAV, and lentiviruses, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

An “antagonist” refers to a substance that interferes with or inhibits the physiological action of another. Therefore, an “immune checkpoint antagonist” this will encompass any substance that interferes with the physiological action of an immune checkpoint-associated protein in a patient in which such antagonist is delivered.

A “target peptide or nucleic acid composition” refers to a composition comprising a peptide (or polymer comprising amino acids as defined above) of the present invention, which can be the immune checkpoint antagonist and/or the antigen of the composition, or a nucleic acid sequence encoding for that target peptide. Therefore, a “target peptide or nucleic acid composition” refers to the antagonist and/or antigen either in peptide form or nucleic acid form.

Immune Checkpoint Antagonist

Immune checkpoint-associated proteins regulate immune activation of certain immune system cells, such as T cells. Checkpoint-associated proteins help keep immune responses in check and can prevent such immune system cells from killing infected or cancerous cells. However, inhibitors of immune checkpoint-associated proteins can block and inhibit the immune system suppression, thereby allowing the T cells to better attack and kill infected or cancer cells.

Immune checkpoint-associated proteins may include ligands or their respective receptors. For example, immune checkpoint-associated proteins include, but are not limited to, CTLA-4, PD-L1, PD-L2, CD80, CD86, ICOS Ligand, B7.1/2, B7-H3, B7-H4, 4-1BBL, HVEM, MHC-Class I, MHC Class II, OX40L, CD70, CD40L, Galectin-9, Adenosine, GITRL, IDO, TDO, CEACAM1, VISTA, CD40, CD47, CD155, CD48, HHLA2, BTN2A1, BTN2A2, BTN3A1, BTNL3, BTNL9, PD-1, CTLA-4, CD28, ICOS, 4-1BB, BTLA, CD160, LIGHT, LAG3, OX40, CD27, CD40, TIM-3, Adenosine A2a receptor, GITR, CEACAM1, SIPR-alpha, DNAM-1, TIGIT, CD96, 2B4, TMIGD2, DC-SIGN, 4.1BBL, A2Ar, BTNL2, CD30, CD244, CSFIR, CXCR4-CXCL12,ICOSL, IgSF, ILTs, LIK, MICA/MICB, Neuropilin,NKG2A, phosphatidylserine, Siglec3, TGF-B, TLIA, and TNFRSF25.

In some embodiments of the invention, the immune checkpoint-associated protein is a ligand such as PD-L1, PD-L2, CD80, CD86, ICOS Ligand, B7-H3, B7-H4, 4-1BBL, HVEM, MHC-Class I, MHC Class II, OX40L, CD70, CD40L, Galectin-9, Adenosine, GITRL, IDO, TDO, CEACAM1, VISTA, CD47, CD155, CD48, HHLA2, BTN2A1, BTN2A2, BTN3A1, BTNL3 and BTNL9. In another aspect of the invention, the immune checkpoint-associated protein is a soluble receptor, including but not limited to PD-1, CTLA-4, CD28, ICOS, 4-1BB, BTLA, CD160, LIGHT, LAG3, OX40, CD27, CD40, TIM-3, Adenosine A2a receptor, GITR, CEACAM1, SIPR-alpha, DNAM-1, TIGIT, CD96, 2B4, TMIGD2, and DC-SIGN.

The immune checkpoint antagonist or inhibitor interacts with the immune checkpoint-associated protein in such a way as to reduce, interfere with, impair, or otherwise eliminate the ability of the checkpoint protein to be expressed or function. The antagonist may act by competitive binding to the ligand or receptor, or otherwise interfering with the expression of the immune checkpoint associate protein. Preferably, the antagonist is a knockout component against the target immune checkpoint. The antagonist may be a biologic therapeutic or small molecule. For example, the antagonist may be a peptide. The peptide may be delivered in the form of a small molecule/polypeptide or as a vector encoding the peptide or coated onto a nano/micro-particles. In some embodiments, the immune checkpoint antagonist is a monoclonal antibody, a humanized antibody, a fully human antibody, a fusion protein, or a combination thereof specific for immune checkpoint associated proteins. In another embodiment, the antagonist is a protein that competes with the binding of the ligand or receptor.

Suitable antagonists include, but are not limited to, CTLA-4 antagonist, vermurafenib, ipilimumab, dacarbazine, IL-2, temozolomide, imatinib, gefitinib, erlotinib, sunitinib, tyrphostins, a PD-1 agonist/antagonist, and telatinib.

In some aspects of the invention, the antagonist is delivered to a patient in need thereof in the form of a nucleic acid. In other embodiments of the invention, the nucleic acid is codon optimized to yield a more stable mRNA than one encoding the same protein, which has not been so optimized. The antagonist may be delivered in a viral or nonviral vector. In one embodiment, the vector is a naked DNA, plasmid, a liposome, or nanoparticles. In another embodiment, the vector is a viral vector, such as an adenovirus, adeno-associated virus (AAV), retrovirus, herpes simplex virus, or alphavirus. In yet another embodiment, the vector is a combination of a viral and a nonviral vector, such as delivery of an AAV virus in a liposome. The viral and non-viral delivery vehicles of the present invention can be made using techniques well known to those skilled in the art. Such techniques are readily available in, for example, Sambrook, Fritsch and Maniatis, Molecule Cloning: A Laboratory Manual, Third Edition (1994); Ausubel et al., Current Protocols in Molecular Biology (1992); Miller and Calos (editors), Gene Transfer Vectors for Mammalian Cells (1987); Kriegler, Gene Transfer and Expression: A Laboratory Manual (1991), all incorporated herein by reference.

Delivery of the antagonist as a nucleic acid may include a system to allow for a knockout methods, such as gene editing of a patient's cells. For example, in one embodiment, a composition of the present invention may further include the CRISPR-Cas9 system. In this embodiment, the composition comprising the nucleic acid encoding the antagonist further comprises a nucleic acid encoding the Cas9 enzyme, optionally having a transcriptional regulator attached thereto, in combination with a guide RNA. The guide RNA facilitates the enzymatic activity of the Cas9 protein, as is known in the art, and shown, for example, in WO2017023974, herein incorporated by reference. The composition may alternatively further comprise a nucleic acid that is trans-activating crRNA (tracrRNA) or CRISPR RNAs (crRNA) or a combination thereof Alternatively, the knockout methods may include siRNA, dsRNA, microRNA, ribozyme, TALENs (transcription activator-like effector nuclease), and ZFNS (zinc finger nuclease).

The antagonist is delivered in an amount effective to enhance the priming of an immune response, enhance the effectiveness of an immune response, and/or obtain overexpression in APCs to induce immunity against the antigen present in the composition. Therefore, the antagonist will modulate the immune checkpoint-associated proteins to release immune suppression or activate the immune system elicited, e.g., by auto tissues, tumors against immune effector (T) cells. In the case of neoantigens in combination with checkpoint antagonists, the composition will downregulate the activity of the immune checkpoint-associated proteins and (1) elicit an immune recognition or priming for the neoantigen epitopes; (2) expand the abundance of neoantigen specific T memory cell clones; and (3) increase the ratio of anti-neoantigen T cell clones in entire T cell repertoires, either by expanding the memory T cells for the specific antigen already in the patient or by priming the naive T cell against the neoantigens. In the case of autoantigens in combination with checkpoint antagonists, the composition will upregulate the activity of checkpoint-associated proteins within APCs to induce immune tolerance for the autoantigen epitopes.

II Antigens

The present invention is directed to a composition further comprising an antigen or an antigenic peptide (e.g., epitope). Preferably, the antigen or antigenic peptide is recognized by autologous T cells. Any antigen may be used in the present invention that is displayed or detected on the surface of tumorous or infected cells. Such antigens include both foreign and self antigens. In many cases, a patient will recognize such antigens a “non-self” or foreign. The antigen may be a wild type antigen or mutated relative to its wild type; or may be differentially post-translationally modified relative to the wild type.

In accordance with the present invention, the antigen may be a self-antigen or foreign antigen. It an embodiment of the invention, the antigen is a tumor-associated antigen, such as a cancer-testes associated antigen. The antigen may be a neoantigen, and specifically a cancer neoantigen. Cancer neoantigens are tumor-specific antigens generated from gene mutations occurring in tumor cells. There are patient-specific somatic mutations occurring during neoplastic transformation and are particularly useful in the present invention.

Antigens recognized by T cells, whether helper T lymphocytes or CTL, are not recognized as intact proteins, but rather as small peptides that associate with class I or class II MHC proteins on the surface of cells. During the course of a naturally occurring immune response, antigens that are recognized in association with class I or II MHC molecules on antigen presenting cells (APCs) are acquired from outside the cell, internalized, and processed into small peptides that associate with the class I or II MHC molecules.

Antigens that give rise to proteins that are recognized in association with class I MHC molecules are generally proteins that are produced within the cells, and these antigens are processed and associate with class I MHC molecules. It is now understood that the peptides that associate with given class I or class II MHC molecules are characterized as having a common binding motif, and the binding motifs for a large number of different class I and II MHC molecules have been determined. Synthetic peptides can also be synthesized that correspond to the amino acid sequence of a given antigen and that contain a binding motif for a given class I or II MHC molecule. These peptides can then be added to appropriate APCs, and the APCs can be used to stimulate a T helper cell or CTL response either in vitro or in vivo. The binding motifs, methods for synthesizing the peptides, and methods for stimulating a T helper cell or CTL response are all known and readily available to one of ordinary skill in the art.

In an embodiment of the invention, the antigen is a peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, beta-Catenin, CDK4, Mum-1, p16, TAGE, PSMA, PSCA, CT7, telomerase, 43-9F, 5T4, 791Tgp72, alpha-fetoprotein, beta-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, b-amyloid, CA125, CD40, EGFR, G17DT, GD2/3L, gp100, IMA950, KOC1, Peptidyl arginine deiminase-4, MUC-1, OFA, PANVAC, PAP, PSA, PSMA, SL701, SSX-2, TTK, TACAS, URLC10, vEGFR, WT-1, BRAF, Pseudomona Exotoxin A, or Diphetheria toxin. In one embodiment, the antigen is selected from patient specific neoantigens or b-amyloid protein or tumor antigen with high mutation loads.

In another embodiment, the antigen is present on the cancer cells of a patient suffering from cancer, such as melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, ovarian cancer, and cervical cancer.

In a particular embodiment of the present invention, the antigen is a weak antigen whose immune response is increased when combined in the presence of the checkpoint antagonist. For example, the antigen may be HER-2/neu in a method of treating breast cancer. Examples of such HER-2/neu antigen are publicly available and include mutations of HER-2/neu including, but not limited to, the HER-2 kinase domain mutations G309A, D769H, D769Y, V777L, P780ins, V842I and R896C mutations. In another embodiment, the antigen is a cytotoxic protein, such as Pseudomonas Exotoxin A or Diphtheria toxin in a method of treating autoimmune reactions. In another embodiment, the antigent is the beta-amyloid antigen in a method of treating or preventing Alzheimer's disease and cancers. Another embodiment is directed to using an auto-antibody against PDL1 and guide RNA targeting PDL1 and/or CTLA-4 in a method of treating diseases such as bladder cancer and non-small cell lung cancer.

In another embodiment, the antigen may be specific for an automimmune disease. In such case, the antigen may be associated with systemic lupus erythematosus, Sjogren's syndrome, SLE, Mixed connective tissue disease, Primary billary cirrhosis, coeliac disease, rheumatoid arthritis, myasthenia Gravis, scleromyositis, Graves disease, Hashimoto's thyroiditis, paraneoplastic cerebellar degeneration, limbic encephalitis, encephalomyelitis, choreathetosis, subacute sensory neuronopathy, stiff person syndrome, diabetes mellitus type 1, optic neuropathy, chorea, or Devics syndrome (neuromyelitis optica).

Another embodiment includes the use of antigens associated with transplant rejection. In this manner, the transplantation rejection-related antigen includes, but are not limited to, all the donor unmatched HLA types according to publicly available computer algorithms that determine HLA matching at the epitope level (e.g., HLAMatchmaker), such as HLA AI (Eplet163RG), A1+A36 (44KM₂), A2 (66RKH), A2+A28 (142MT), A2+A69 (107W), A3 (161D), A9 (66GKH), A10 (149TAH), A11 (151AHA), A25+A32 (76ASI), A29 (62LQ), A30 (152RW), A30+A31(56R), A31+A33 (73ID), A68 (245VA), B5+B35 (193PV), B7 (177DK), B8+Cw7 (9D), B12 (167ES), B13 (144QL), B15 (163LW), B16 (158T), B17 (71SA), B18 (30G), B18+B35 (44RT), B27 (71KA), B40 (44RK), B44 (199V),B48 (245TA), Bw4 (82LR), Bw6 (80ERN), Cwl (6K), Cw2 (211T), Cw3 (173K), Cw4 (17WR), DR51 (96EN₃), Cw5+8 (138K), Cw7 (193PL), DR1 (12LKF₂), DR1+10 (13FEL), DR1+51 (96EV), DR2 (142M₂), DR3 (74R), DR4 (96Y2), DR3+6 (31YYFH), DR7 (25Q₃), DR7+9 (78V2), DR8 (25YRF), DR8+12 (16Y), DR9 (13FEY), R10 (40YD₂), DR11 (57DE), DR12 (37L), DR13 (71DEA), DR14 (57AA), DR15 (71A), DR17 (26TYD), DR51 (96EN₃), DR52 (98Q), DR53 (48YQ6), DQ7+9 (56PPD), DQ8 (56PPA), DQ7 (45EV), DQ6 (125G), DQ5 (74SR₃), DQ4 (56L₂), DQ3 MICA,(55PP), DQ2 (45GE₃), DQ1 (52PQ₂). Additionally, non-HLA antigens that may be used in the present invention include, but are not limited to, VACM1, ICAM1, Vimentin, Cardiac Myosin, Collagen V, k-α1 tubulin, and angiotensin II receptor type I.

Additional suitable autoantigens include, but are not limited to histidine-tRNA ligase, Ribonucleoproteins, snRNP core proteins, type I topoisomerase, histones, nucleoporin62, Sp100 nuclear antigen, nucleoporin 210Kda, actin, cyclic citrullinated peptide, thrombin, exosome complex proteins, nicotine acetylcholine receptor, muscle specific kinase, voltage-gated calcium channel, thyroid peroxidase, thyroglobulin, TSH receptor, Neuronal nuclear proteins, glutamate receptor, amphiphysin, glutamate decarboxylase, collapsing response mediator protein 5, N-methyl-D-aspartate receptor, and aquaporin. The present invention further includes the use antigen recognized by individual specific autoantibody (ISA) as identified using antibody profiling technology, as known in the art. In another embodiment, the invention includes donor antigens related to organ transplantation rejection.

In accordance with one embodiment, the compositions of the present invention include an antigen nucleotide sequence encoding the antigen or epitope. Accordingly, the nucleotide sequence encoding the antigen may be present on a vector that is delivered to a patient along with the delivery of the immune checkpoint antagonist. Notably, one or more antigenic peptides may be delivered in the composition.

In another embodiment, the antigen may comprise at least one neoantigen polypeptide that is mutated relative to wild type. This mutant polypeptide may bind to an MHC-Class I or MHC-Class II molecule.

Neoantigens within the scope of the invention may be present across certain patient populations and tumor types. These are patient-specific mutations and therefore, the present invention encompasses the identification of neoantigens for individual patients. Optionally, it is encoded by an oncogene. In one embodiment, the antigen is recognized by a T Cell Receptor (TCR). In some embodiments, the mutation relative to wild type in the neoantigen polypeptide is optionally identified by massive parallel sequencing, epitope prediction algorithms, or in vitro T-cell assays for epitope validation, as is known in the art. Sequencing may be conducted by sequencing a portion of a tumor cell's genome and comparing that tumor sequence to a corresponding portion of a healthy donor cell's genome. Preferably, the sequence is done by next generation sequencing (NGS).

III. Compositions

The compositions of the presently disclosed subject matter can in some embodiments include about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50-55, 55-65, 65-80, 80-120, 90-150, 100-175, or 175-250 different checkpoint antagonists and/or antigens.

The phrase “antagonist/antigen compositions” as used herein refers to at least one checkpoint antagonist to an immune checkpoint-associated protein in combination with an antigen of the present invention formulated, for example, as an immunogenic composition, such as a vaccine; or as a preparation for pulsing cells in a manner such that the pulsed cells, e.g., dendritic cells, will display the at least one antigenic peptide of the composition on their surface, e.g., to T cells in the context of adoptive T cell therapy. The checkpoint antagonist and antigen can be peptides, which may be the same or different. The antagonist may be delivered as a small molecule, a nucleic acid, peptide, or a protein provided that it acts to diminish, impair, or otherwise disrupt the function or expression of an immune checkpoint-associated protein. The antigen may be delivered as a nucleic acid, peptide, or a protein. The antigen is selected to modulate, e.g., the upregulate or downregulate, the immune system of a patient in combination with the antagonist. In some embodiments, the immune systems is primed, induced, or enhanced in response to the antigen in combination with the immune checkpoint antagonist.

The composition of the present invention encompasses any variations of such antagonist/antigen compositions. Preferably, the antagonist/antigen composition is a nucleic acid. In another embodiment, the antagonist/antigen composition is delivered in one or more vectors.

The compositions of the presently disclosed subject matter in some embodiments may include WIC class I specific peptide as an antigen, but can also include one or more antigenic peptides specific for MHC class II, or other peptides associated with tumors (e.g., tumor associated antigen (“TAA”)) such as, but not limited to those disclosed in Table 2.

Compositions comprising the antigen peptide are can be made synthetically or by purification from a biological source. They can be made recombinantly. Desirably they are in some embodiments at least 90% pure, in some embodiments at least 92% pure, in some embodiments at least 93% pure, in some embodiments at least 94% pure, in some embodiments at least 95% pure, in some embodiments at least 96% pure, in some embodiments at least 97% pure, in some embodiments at least 98% pure, and in some embodiments at least 99% pure. For administration to a human, they generally do not contain other components that might be harmful to a human recipient (referred to herein as “pharmaceutically acceptable for use in a human”). The compositions are typically devoid of cells, both human and recombinant producing cells. However, as noted below, in some cases, it can be desirable to load dendritic cells with a target peptide and use those loaded dendritic cells as either an immunotherapy agent themselves or as a reagent to stimulate a patient's T cells ex vivo. The stimulated T cells can be used as an immunotherapy agent.

Under certain circumstances it can be desirable to add additional antigenic proteins or antigenic peptides to the composition, for example, to make a cocktail having the ability to stimulate an immune response in a number of different HLA type hosts. Alternatively, additional proteins and/or peptides can provide an interacting function within a single host, such as but not limited to an adjuvant function or a stabilizing function. As a non-limiting example, tumor antigens can be used in admixture with the antigen peptides such that multiple different immune responses are induced in a single patient.

Administration of antigen peptides to a mammalian recipient can be accomplished using long antigen peptides (e.g., longer than 15 residues), and/or using antigen peptide-loaded dendritic cells. (Melief, 2009.) In some embodiments, an immediate goal of the administration of target peptides is to induce activation of CD8⁺ T cells in a subject. Additional components that can be administered to the same subject, either at the same time and/or close in time (such as but not limited to within 3, 5, 7, 10, 14, 17, or 21 days of each other, or even longer) include TLR-ligand oligonucleotide CpG and related antigen peptides that have overlapping sequences of at least six amino acid residues. To ensure efficacy, mammalian recipients should express the appropriate human HLA molecules to bind to the target peptides. Transgenic mammals can be used as recipients, for example, if they express appropriate human HLA molecules. If a mammal's own immune system recognizes a similar target peptide then it can be used as model system directly without introducing a transgene. Useful models and recipients can be at increased risk of developing metastatic cancer, such as metastatic melanoma. Other useful models and recipients can be predisposed, e.g., genetically and/or environmentally, to develop melanoma or other cancer.

### III.A. Selection of Antagonist and Antigens

Disclosed herein is the finding that immune responses can be enhanced by checkpoint antagonists and antigenic peptides, preferably those found or detected on the surface of cancer cells, as tested in healthy and diseased individuals. The T cells associated with these immune responses are able to recognize and kill malignant tissue (both established cells lines and primary tumor samples).

When selecting antigens of the presently disclosed subject matter for inclusion in immunotherapy, e.g., in adaptive cell therapy or in the context of a vaccine, one can in some embodiments pick target peptides using one or more of the following criteria: 1) peptides associated with a particular disease/transplant rejection/cancer/tumor cell type; 2) a peptide derived from a gene product (e.g., a polypeptide) associated with cell proliferation, transformation, and/or malignancy; 3) a peptide that is specific for an HLA allele carried the group of patients to be treated; and/or 4) a peptide that is capable of inducing a target peptide-specific memory T cell response in the patients to be treated upon a first exposure to a composition including the selected target peptides.

### III.B. Antigen Peptide Vaccines

The checkpoint antagonist and antigen peptides and nucleic acids encoding the antagonist and/or antigen can also be employed in a composition designed to vaccinate an individual. These peptides are delivered together either simultaneously or sequentially. Also, it is possible that the antagonist is delivered as a small molecule, peptide, protein, or nucleic acid encoding a peptide or protein, whereas the antigen is delivered as a peptide, protein, or nucleic acid encoding an antigenic peptide or protein. In some embodiments, the antagonist and antigenic peptide are injected individually or, alone and can in some embodiments be administered in combination with an adjuvant and/or a pharmaceutically acceptable carrier. Vaccines are envisioned to prevent and/or treat certain diseases in general, and cancers in particular.

The antagonist/antigen compositions of the presently disclosed subject matter can in some embodiments be used as a vaccine for cancer, and more specifically for melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, ovarian cancer, and cervical cancer. Alternatively, the vaccines may be used to protect against organ transplantation, infectious diseases, or autoimmune diseases.

The compositions include antigen peptides or nucleic acids encoding the antigen peptides. The vaccine compositions of the present invention, in some embodiments, include the antigen peptides, or peptides disclosed herein, in addition to other cancer antigens that have been identified. The compositions may include one or more antigen peptides.

The vaccine compositions of the presently disclosed subject matter can be used prophylactically for the purposes of preventing, reducing the risk of, and/or delaying initiation of a disease in an individual that does not currently have the disease. Alternatively, they can be used to treat an individual that already has the disease, so that recurrence or development of the disease is delayed and/or prevented. Prevention relates to a process of prophylaxis in which the individual is immunized prior to the induction or onset of the disease. For example, in some embodiments, at-risk individuals with a history of the disease can be immunized prior to the onset of the disease.

Alternatively, individuals that already have disease can be immunized with the antigen-containing compositions of the presently disclosed subject matter so as to stimulate an immune response that would be reactive against the disease. A clinically relevant immune response would be one in which the disease partially or completely regresses and is eliminated from the patient, and it would also include those responses in which the progression of the disease is blocked without being eliminated. Similarly, prevention need not be total, but may result in a reduced risk, delayed onset, or delayed progression or metastasis.

In some embodiments, the 5-year survival rate of patients treated with the vaccines of the presently disclosed subject matter is increased by a statistically significant amount: e.g., by about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent, or even greater than 100 percent, relative to the average 5-year survival rates.

In some embodiments, the compositions of the presently disclosed subject matter increase survival rates in patients with metastatic cancer by a statistically significant amount of time such as, but not limited to by about or at least 0.25, 0.5, 0.75, 1.0, 1.25, 1.5, 1.75, 2.0, 2.25, 2.5, 2.75, 3.0, 3.25, 3.5, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.50, 9.75, 10.0, 10.25, 10.5, 10.75, 11.0, 11.25, 11.5, 11.75, or 12 months or more compared to what could have been expected without treatment at the time of filing of this specification.

In some embodiments, the survival rate (e.g., the 1, 2, 3, 4, or 5-year survival rate) of patients treated with the compositions of the presently disclosed subject matter is increased by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent, or even greater than 100 percent, relative to the average 5-year survival rates.

The compositions of the presently disclosed subject matter are in some embodiments envisioned to illicit a T cell-associated immune response such as, but not limited to generating activated CD8⁺ T cells specific for an antigenic peptide/MHC class I expressing cells. In some embodiments, the CD8⁺ T cells specific for native target peptide/MHC class I expressing cells are specific for at least or about 1, 2, 3, 4, 5, 6, 7, 8, 9, 10, or more of the target peptides in the composition in a patient for about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more days after providing the composition to the patient.

In some embodiments, the treatment response rates of patients treated with the compositions of the presently disclosed subject matter are increased by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 07, 98, 99, 100, 150, 200, 250, 300, 350, 400, 450, or 500 or more percent, relative to treatment without the composition.

In some embodiments, overall median survival of patients treated with the compositions of the presently disclosed subject matter is increased by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, or 500 or more percent, relative to treatment without the composition. In some embodiments, the overall median survival of cancer patients treated with the compositions is envisioned to be about or at least 10.0, 10.25, 10.5, 10.75, 11.0, 11.25, 11.5, 11.75, 12, 12.25, 12.5, 12.75, 13, 13.25, 13.5, 13.75, 14, 14.25, 14.5, 14.75, 15, 16, 17, 18, 19, 20, 21, 22, 23, 24, 25, 26, 27, 28, 29, 30, 31, 32, 33, 34, 35, 36 or more months.

In some embodiments, tumor size of patients treated with the compositions of the presently disclosed subject matter is decreased by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, or 500 or more percent, relative to treatment without the composition.

In some embodiments, the compositions of the presently disclosed subject matter provide a clinical tumor regression that is by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of patients treated with the composition.

In some embodiments, the compositions of the presently disclosed subject matter provide a CTL response specific for the cancer being treated, e.g., melanoma, by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of patients treated with the composition.

In some embodiments, the compositions of the presently disclosed subject matter provide an increase in progression free survival in the cancer being treated, such as but not limited to melanoma, of about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500 or more months compared to the progression free survival or patients not treated with the composition.

In some embodiments, one or more of progression free survival, CTL response rates, clinical tumor regression rates, tumor size, survival rates (such as but not limited to overall survival rates), and/or response rates are determined, assessed, calculated, and/or estimated weekly, monthly, bi-monthly, quarterly, semi-annually, annually, and/or bi-annually over a period of about or at least 1, 2, 3, 4, 5, 6, 7, 8, 9, 10, 11, 12, 13, 14, 15 or more years or about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500 or more weeks.

### III.C. Compositions for Priming T Cells

Adoptive cell transfer (ACT) is the passive transfer of cells, in some embodiments immune-derived cells, into a recipient host with the goal of transferring the immunologic functionality and characteristics into the host. Clinically, this approach has been exploited to transfer either immune-promoting or tolerogenic cells (often lymphocytes) to patients to enhance immunity against cancer. The adoptive transfer of autologous tumor infiltrating lymphocytes (TIL) or genetically redirected peripheral blood mononuclear cells has been used to successfully treat patients with advanced solid tumors, including melanoma and colorectal carcinoma, as well as patients with CD19-expressing hematologic malignancies. In some embodiments, ACT therapies achieve T cell stimulation ex vivo by activating and expanding autologous tumor-reactive T cell populations to large numbers of cells that are then transferred back to the patient. See Gattinoni et al., 2006.

The antagonists and antigens of the presently disclosed subject matter can in some embodiments take the form of antigenic peptides formulated in a composition added to autologous dendritic cells and used to stimulate a T helper cell or CTL response in vitro. The in vitro generated T helper cells or CTL can then be infused into a patient with cancer (Yee et al., 2002), and specifically a patient with a form of cancer that expresses one or more of antigenic peptides.

Alternatively, the peptides can be added to dendritic cells (DCs) in vitro to produce loaded DCs, with the loaded DCs being subsequently transferred into an individual with cancer in order to stimulate an immune response. Alternatively, the loaded DCs can be used to stimulate CD8⁺ T cells ex vivo with subsequent reintroduction of the stimulated T cells to the patient. Although a particular antigen might be identified on one particular cancer cell type, it might also be found on other cancer cell types.

The presently disclosed subject matter envisions treating cancer by providing a patient with cells pulsed with a composition of target peptides. The use of DCs pulsed with antigen peptide enables manipulation of the immunogen in two ways: varying the number of cells injected and varying the density of antigen presented on each cell. Exemplary non-limiting methods for DC-based treatments can be found, for example in Mackensen et al., 2000.

### III.D. Additional Peptides Present in Antagonist/Antigen Compositions

The antagonist/antigen compositions (or antigen peptide composition kits comprising the same) of the presently disclosed subject matter can in some embodiments also include at least one additional peptide derived from one or more tumor-associated antigens (TAAs). Examples of TAAs include MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAIVIE, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY—CO-1, RCAS1, SDCCAG16, TA-90 (Mac-2 binding protein/cyclophilin C-associated protein), TAAL6, TAG72, TLP, TPS, prostatic acid phosphatase, and the like. Exemplary, non-limiting peptides derived from TAAs that can be incorporated into target peptide compositions (or target peptide composition kits comprising the same) of the presently disclosed subject matter are presented in Table 1.

TABLE 1
gp100280-288 YLEPGPVTA (SEQ ID NO: 1)
Tyr369-377 DYMDGTMSQV (SEQ ID NO: 2)
gp10017-25 ALLAVGATK (SEQ ID NO: 3)
Tyr243-251 KCDICTDEY (SEQ ID NO: 4)
TAG-1,2 RLSNRLLLR (SEQ ID NO: 5)
SQNFPGSQK (SEQ ID NO: 6)
gp100154-162 KTWGQYWQV (SEQ ID NO: 7)
gp100209-217 I(T/M)DQVPFSV (SEQ ID NO: 8)
gp100476-485 VLYRYGSFSV (SEQ ID NO: 9)
MART-1/MelanA27-35 AAGIGILTV (SEQ ID NO: 10)
gp100 ALNFPGSQK (SEQ ID NO: 11)
gp100614-622 LIYRRRLMK (SEQ ID NO: 12)
NY-ESO-1 AAQERRVPR (SEQ ID NO: 13)
NY-ESO-1 ASGPGGGAPR (SEQ ID NO: 14)
NY-ESO-1 LLGPGRPYR (SEQ ID NO: 15)
Tyr240-251 SDAEKSDICTDEY (SEQ ID NO: 16)
Tyr146-156 SSDYVIPIGTY (SEQ ID NO: 17)
MAGE-A1161-169 EADPTGHSY (SEQ ID NO: 18)
MAGE-A3168-176 EVDPIGHLY (SEQ ID NO: 19)
Tyr369-377 D YMDGTMSQV (SEQ ID NO: 20)
gp100209-217 IMDQVPFSV (SEQ ID NO: 21)
gp100280-288 YLEPGPVTA (SEQ ID NO: 22)
MAGE-A10254-262 GLYDGMEHL (SEQ ID NO: 23)
gp100614-622 LIYRRRLMK (SEQ ID NO: 24)
NY-ESO-153-62 ASGPGGGAPR (SEQ ID NO: 25)
Tyr56-70 AQNILLSNAPLGPQFP (SEQ ID NO: 26)
Tyr388-406 FLLHHAFVDSIFEQWLQRHRP (SEQ ID NO: 27)
Melan-A/MART-151-73 RNGYRALMDKSLHVGTQCALTRR
(SEQ ID NO: 28)
MAGE-A3281-295 TSYVKVLHEIMVKISG (SEQ ID NO: 29)
MAGE-A1,2,3,6121-134 LLKYRAREPVTKAE
(SEQ ID NO: 30)
Gp10044-59 WNRQLYPEWTEAQRLD (SEQ ID NO: 31)
Her2/neu369-377 KIFGSLAFL (SEQ ID NO: 32)
p2830-844 AQYIKANSKFIGITEL (SEQ ID NO: 33)
CEA571-579 YLSGADLNL (SEQ ID NO: 34)
Her2/neu754-762 VLRENTSPK (SEQ ID NO: 35)
MAGE-A1161-169 EADPTGHSY (SEQ ID NO: 36)
FBP191-199 EIWTHSYKV (SEQ ID NO: 37)
MAGE-A196-104 SLFRAVITK (SEQ ID NO: 38)
MAGE-A3168-176 EVDPIGHLY (SEQ ID NO: 39)
MAGE-A10254-262 GLYDGMEHL (SEQ ID NO: 40)
CEA27-35 HLFGYSWYK (SEQ ID NO: 41)
NY-ESO-153-62 ASGPGGGAPR (SEQ ID NO: 42)
MART-197-116 APPAYEKLS (SEQ ID NO: 43)
MART-198-109 PPAYEKLSA (SEQ ID NO: 44)
MART-199-110 PAYEKLSAE (SEQ ID NO: 45)
MART-197-116 VPNAPPAYEKLpSAEQSPPPY (SEQ ID NO: 46)
MART-198-109 PNAPPAYEKLpSA (SEQ ID NO: 47)
MART-199-110 NAPPAYEKLpSAE (SEQ ID NO: 48)
MART-1100-111 APPAYEKLpSAEQ (SEQ ID NO: 49)
MART-1100-114 APPAYEKLpSAEQSPP (SEQ ID NO: 50)
MART-1100-115 APPAYEKLpSAEQSPPP (SEQ ID NO: 51)
MART-1100-116 APPAYEKLpSAEQSPPP (SEQ ID NO: 52)
MART-1101-112 PPAYEKLpSAEQS (SEQ ID NO: 53)
MART-1102-113 PAYEKLpSAEQSP (SEQ ID NO: 54)
MART-1103-114 AYEKLpSAEQSPP (SEQ ID NO: 55)
MART-1104-115 YEKLSAEQSPPP (SEQ ID NO: 56)
MART-1100-111 APPAYEKLpSAEQ (SEQ ID NO: 57)
MART-1100-114 APPAYEKLpSAEQSPP (SEQ ID NO: 58)
MART-1100-115 APPAYEKLSAEQSPPP (SEQ ID NO: 59)
¹the numbers listed in lowercase denote the amino acid positions of the peptide sequences for each TAA.

Such tumor-specific peptides may be the target antigen peptide of the present invention, present in a manner, number, and in an amount as if they were an additional target peptide added to the target peptide compositions as described herein.

### III.E. Combination Therapies

In some embodiments, the compositions or kit of the present invention are administered as a vaccine or in the form of pulsed cells as first, second, third, or fourth line treatment for the diseases, including Alzheimer's, cancer, autoimmune diseases, and transplant rejection. In some embodiments, the compositions of the presently disclosed subject matter are administered to a patient in combination with one or more therapeutic agents. Exemplary, non-limiting therapeutic agents include anti-Programmed Death-1 (PD1) or PD 1 -antagoni sts such as the anti-PD1 antibody BM S-93 655 8 (Bristol-Myers Squibb Co., New York, N.Y., United States of America); anti-CTLA-4 or CTLA-4 antagonists; vermurafenib; ipilimumab; Dacarbazine; IL-2; Temozolomide; receptor tyrosine kinase inhibitors, including but not limited to imatinib, gefitinib, erlotinib, sunitinib, tyrphostins, telatinib; sipileucel-T; a platinum-based agent; a taxane; an alkylating agent; an antimetabolite and/or a vinca alkaloid; and combinations thereof.

In some embodiments, the cancer is sensitive to and/or refractory, relapsed, and/or resistant to one or more chemotherapeutic agents such as, but not limited to a platinum-based agent, a taxane, an alkylating agent, an anthracycline (e.g., doxorubicin including but not limited to liposomal doxorubicin), an antimetabolite, and/or a vinca alkaloid. In some embodiments, the cancer is an ovarian cancer, and the ovarian cancer is refractory, relapsed, or resistant to a platinum-based agent (e.g., carboplatin, cisplatin, oxaliplatin), a taxane (e.g., paclitaxel, docetaxel, larotaxel, cabazitaxel), and/or an anthracycline (e.g., doxorubicin including but not limited to liposomal doxorubicin). In some embodiments, the cancer is colorectal cancer, and the cancer is refractory, relapsed, or resistant to an antimetabolite (e.g., an antifolate (e.g., pemetrexed, floxuridine, raltitrexed) a pyrimidine analogue (e.g., capecitabine, cytrarabine, gemcitabine, 5FU)), and/or a platinum-based agent (e.g., carboplatin, cisplatin, oxaliplatin). In some embodiments, the cancer is lung cancer, and the cancer is refractory, relapsed, or resistant to a taxane (e.g., paclitaxel, docetaxel, larotaxel, cabazitaxel), a platinum-based agent (e.g., carboplatin, cisplatin, oxaliplatin), a vinca alkaloid (e.g., vinblastine, vincristine, vindesine, vinorelbine), a vascular endothelial growth factor (VEGF) pathway inhibitor, an epidermal growth factor (EGF) pathway inhibitor) and/or an antimetabolite (e.g., an antifolate incuding but note limited to pemetrexed, floxuridine, or raltitrexed), and a pyrimidine analogue (e.g., capecitabine, cytrarabine, gemcitabine, 5FU). In some embodiments, the cancer is breast cancer, and the cancer is refractory, relapsed, or resistant to a taxane (e.g., paclitaxel, docetaxel, larotaxel, cabazitaxel), a VEGF pathway inhibitor, an anthracycline (e.g., daunorubicin, doxorubicin including but not limited to liposomal doxorubicin, epirubicin, valrubicin, idarubicin), a platinum-based agent (e.g., carboplatin, cisplatin, oxaliplatin), and/or an antimetabolite (e.g., an antifolate including but not limited to pemetrexed, floxuridine, or raltitrexed), and a pyrimidine analogue (e.g., capecitabine, cytrarabine, gemcitabine, 5FU). In some embodiments, the cancer is gastric cancer, and the cancer is refractory, relapsed, or resistant to an antimetabolite (e.g., an antifolate including but not limited to pemetrexed, floxuridine, raltitrexed) and a pyrimidine analogue (e.g., capecitabine, cytrarabine, gemcitabine, 5FU) and/or a platinum-based agent (e.g., carboplatin, cisplatin, oxaliplatin).

Single-agent dacarbazine (DTIC) treatment in advanced-stage malignant melanoma generally yields only a 10-15% response rate. (Fecher & Flaherty, 2009). Two combination regimens commonly are used in the treatment of patients with advanced-stage melanoma. The first regimen is the cisplatin, vinblastine, and DTIC (CVD) regimen. The second commonly used regimen is the Dartmouth regimen, which is a combination of cisplatin, DTIC, carmustine, and tamoxifen. Among patients with advanced melanoma who had alternations in the type III transmembrane receptor tyrosine kinase KIT, treatment with imatinib mesylate resulted in clinically significant response in a subset of patients (Carvajal et al., 2011). DTIC was the first drug approved for the treatment of metastatic melanoma. In the initial studies with dacarbazine, the overall response rate was 22%, with no impact on survival. In a Phase III study of dacarbazine compared with temozolomide, the response rate was 12% versus 13% (Middleton et al., 2000). Carboplatin and paclitaxel have been tested in 2 small Phase II studies, and when used in combination with sorafenib, the response rate was 11-17%. In some embodiments, temozolomide is included in a first-line drug for melanoma.

In some embodiments, the compositions of the presently disclosed subject matter may be associated with agents that inhibit T cell apoptosis or anergy thus potentiating a T cell response (referred to herein interchangeably as a “T cell potentiator” or “checkpoint antagonist”). Such agents include B7RP1 agonists, B7-H3 antagonists, B7-H4 antagonists, HVEM antagonists, HVEM antagonists, GALS antagonists or alternatively CD27 agonists, OX40 agonists, CD137 agonists, BTLA agonists, ICOS agonists CD28 agonists, or soluble versions of PDL1, PDL2, CD80, CD96, B7RP1, CD137L, OX40 or CD70. See Pardoll, 2012.

In some embodiments, the T cell potentiator is a PD1 antagonist. Programmed death 1 (PD1) is a key immune checkpoint receptor expressed by activated T cells, and it mediates immunosuppression. PD1 functions primarily in peripheral tissues, where T cells can encounter the immunosuppressive PD1 ligands PD-L1 (B7-H1) and PD-L2 (B7-DC), which are expressed by tumor cells, stromal cells, or both. In some embodiments, the anti-PD1 monoclonal antibody BMS-936558 (also known as MDX-1106 and ONO-4538; Bristol-Myers Squibb) is used. In some embodiments, the T cell potentiator (e.g., PD1 antagonist) is administered as an intravenous infusion at least or about every 1, 1.5, 2, 2.5, 3, 3.5, or 4 weeks of each 4, 5, 6, 7, 8, 9, or 10-week treatment cycle of about for at least 1, 2, 3, 4, 5, 6, 7, 8, 9, 10, 11, 12, 13, 14, 15, 16, 17, 18, 19, 20, or more cycles. Exemplary, non-limiting doses of the PD1 antagonists are in some embodiments exactly, about, or at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, or more mg/kg. See Brahmer et al., 2012.

The exemplary therapeutic agents listed herein above are envisioned to be administered at a concentration of in some embodiments about 1 to 100 mg/m², in some embodiments about 10 to 80 mg/m², and in some embodiments about 40 to 60 mg/m². Further exemplary dosages include, but are not limited to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more mg/m².

Alternatively, an exemplary dosage range can be in some embodiments about or at least 0.001 to 100 mg/kg, in some embodiments about or at least 0.1 to 1 mg/kg, and in some embodiments about or at least 0.01 to 10 mg/kg.

The checkpoint antagonist and antigen compositions or kits of the presently disclosed subject matter can in some embodiments be co-administered with cytokines such as lymphokines, monokines, growth factors, and traditional polypeptide hormones. Exemplary cytokines are growth hormones including but not limited to human growth hormone, N-methionyl human growth hormone, and bovine growth hormone; parathyroid hormone; thyroxine; insulin; proinsulin; relaxin; prorelaxin; glycoprotein hormones including but not limited to follicle stimulating hormone (FSH), thyroid stimulating hormone (TSH), and luteinizing hormone (LH); hepatic growth factor; prostaglandin, fibroblast growth factor; prolactin; placental lactogen, OB protein; TNF-.alpha. and TNF-.beta.; mullerian-inhibiting substance; mouse gonadotropin-associated peptide; inhibin; activin; VEGF; integrin; thrombopoietin (TPO); nerve growth factors including but not limited to NGF-beta; platelet-growth factor; transforming growth factors (TGFs) including but not limited to TGF-alpha and TGF-beta; insulin-like growth factor (IGF)-I and IGF-II; erythropoietin (EPO); osteoinductive factors; interferons (IFN) including but not limited to IFNalpha, IFNbeta, and IFNgamma; colony stimulating factors (CSFs) including but not limited to macrophage-CSF (M-CSF), granulocyte-macrophage-CSF (GM-CSF), and granulocyte-CSF (G-CSF); interleukins (ILs) including but not limited to IL-1, IL-1.alpha., IL-2, IL-3, IL-4, IL-5, IL-6, IL-7, IL-8, IL-9, IL-10, IL-11, IL-12; IL-13, IL-14, IL-15, IL-16, IL-17, and IL-18; leukemia inhibitory factor (LIF), kit-ligand; FLT-3; angiostatin; thrombospondin; endostatin; and lymphotoxin (LT). As used herein, the term cytokine includes proteins from natural sources and/or from recombinant cell culture and biologically active equivalents thereof.

The compositions of the presently disclosed subject matter can in some embodiments be provided with administration of cytokines around the time of (including but not limited to about or at least 1, 2, 3, or 4 weeks or days before and/or after) the initial dose of a target peptide composition.

Exemplary non-limiting doses of the cytokine are in some embodiments about or at least 1-100, 10-80, 20-70, 30-60, 40-50, or 1, 2, 3, 4, 5, 6, 7, 8, 9 or 10 Mu/m²/day over about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. The cytokine can in some embodiments be delivered at least or about once every 1, 2, 3, 4, 5, 6, 7, 8, 9, 10, 11, 12, 13, 14, 15, 16, 17, 18, 19, 20, 21, 22, 23, or 24 hours. Cytokine treatment can be provided in at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cycles of at least or about 1, 2, 3, 4, 5, 6, 7, 8, 9, 10, or more weeks, wherein each cycle has at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more cytokine doses. Cytokine treatment can in some embodiments be on the same schedule as administration of the target peptide compositions or in some embodiments on a different schedule, which differing schedule can in some embodiments be an overlapping schedule.

In some embodiments, the cytokine is IL-2 and is dosed in an amount about or at least 100,000 to 1,000,000; 200,000-900,000; 300,000-800,000; 450,000-750,000; 600,000-800,000; or 700,000-800,000 (in some embodiments. 720,000) units (IU)/kg administered, e.g., as a bolus, every 1, 2, 3, 4, 5, 6, 7, 8, 9, 10, 11, 12, 13, 14, 15, 16, 17, 18, 19, or 20 hours for 1, 2, 3, 4, 5, 6, 7, 8, 9, 10, 11, 12, 13, 14, or more days, in a cycle, for example.

### IV. Types of Diseases

In some embodiments, the compositions of the presently disclosed subject matter are envisioned to be useful in the treatment of benign and/or malignant proliferative diseases. Excessive proliferation of cells and turnover of cellular matrix contribute significantly to the pathogenesis of several diseases including but not limited to cancer, atherosclerosis, rheumatoid arthritis, psoriasis, idiopathic pulmonary fibrosis, scleroderma and cirrhosis of the liver, ductal hyperplasia, lobular hyperplasia, papillomas, and others.

In some embodiments, the proliferative disease is cancer, including but not limited to breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer. In some embodiments, the presently disclosed compositions and methods are used to treat melanoma, acute myelogenous leukemia (AML), acute lyphocytic leukemia (ALL), chronic lymphocytic lymphoma (CLL), chronic myelogenous leukemia (CML), breast cancer, renal cancer, pancreatic cancer, and/or ovarian cancer.

In some embodiments, the target peptide compositions of the presently disclosed subject matter can be used to treat melanoma. The melanoma can be in some embodiments Stage I, in some embodiments Stage II (including but not limited to Stages Ha and/or Ilb), Stage III, Stage IV, metastatic, malignant, or recurrent melanoma. When metastatic, the melanoma is in some embodiments in the lung, bone, liver, or brain.

In some embodiments, the cancer is a cancer described herein. For example, the cancer can be a cancer of the bladder (including but not limited to accelerated and metastatic bladder cancer), breast (including but not limited to estrogen receptor positive breast cancer, estrogen receptor negative breast cancer, HER-2 positive breast cancer, HER-2 negative breast cancer, triple negative breast cancer, and inflammatory breast cancer), colon (including but not limited to colorectal cancer), kidney (including but not limited to renal cell carcinoma), liver, lung (including but not limited to small cell lung cancer and non-small cell lung cancer such as but not limited to adenocarcinoma, squamous cell carcinoma, bronchoalveolar carcinoma and large cell carcinoma), genitourinary tract cancer, including but not limited to ovary (such as but not limited to fallopian, endometrial, and peritoneal cancers), cervix, prostate, and testes, lymphatic system, rectum, larynx, pancreas (including but not limited to exocrine pancreatic carcinoma), stomach (including but not limited to gastroesophageal, upper gastric, and lower gastric cancers), gastrointestinal cancer (including but not limited to anal cancer), gall bladder, thyroid, lymphoma (including but not limited to Burkitt's, Hodgkin's, and non-Hodgkin's lymphoma), leukemia (including but not limited to acute myeloid leukemia), Ewing's sarcoma, nasoesophageal cancer, nasopharyngeal cancer, neural and glial cell cancers (including but not limited to glioblastoma multiforme), and head and neck cancers. Exemplary non-limiting cancers also include melanoma, breast cancer (including but not limited to metastatic or locally advanced breast cancer), prostate cancer (including but not limited to hormone refractory prostate cancer), renal cell carcinoma, lung cancer (including but not limited to small cell lung cancer and non-small cell lung cancer (including adenocarcinoma, squamous cell carcinoma, bronchoalveolar carcinoma, and large cell carcinoma), pancreatic cancer, gastric cancer (including but not limited to gastroesophageal, upper gastric, and/or lower gastric cancer), colorectal cancer, squamous cell cancer of the head and neck, ovarian cancer (including but not limited to advanced ovarian cancer, platinum-based agent-resistant, and/or relapsed ovarian cancer), lymphoma (including but not limited to Burkitt's, Hodgkin's, or non-Hodgkin's lymphoma), leukemia (including but not limited to acute myeloid leukemia), and gastrointestinal cancer.

The present compositions may be designed for the treatment or prevention of autoimmune diseases. In such cases, the autoimmune disease may include systemic lupus erythematosus, Sjogren's syndrome, SLE, Mixed connective tissue disease, Primary billary cirrhosis, coeliac disease, rheumatoid arthritis, myasthenia Gravis, scleromyositis, Graves disease, Hashimoto's thyroiditis, paraneoplastic cerebellar degeneration, limbic encephalitis, encephalomyelitis, choreathetosis, subacute sensory neuronopathy, stiff person syndrome, diabetes mellitus type 1, optic neuropathy, chorea, and Devics syndrome (neuromyelitis optica).

The compositions of the presently disclosed subject matter may be used in a treatment or prevention of organ transplantation rejection. In such case, the donor unmatched HLA antigen and/or non-HLA antigens are identified and presented in a composition of the present invention along with a checkpoint antagonist. In such case, inducement of immune tolerance for such unmatched antigen may be attained.

Compositions may further be designed for the treatment or prevention of infectious diseases using antigens specific for such diseases.

Additionally, methods for the treatment of neurodegenerative diseases is within the scope of the present invention. For example, compositions of the present invention may be directed to a method of treating or preventing Alzheimer's disease.

### V. Administration of Compositions

V.A. Routes of Administration

The antagonist/antigen compositions of the presently disclosed subject matter can be administered parenterally, systemically, topically, or any combination thereof. By way of example and not limitation, composition injections can be performed by intravenous (i.v.) injection, subcutaneous (s.c.) injection, intradermal (i.d.) injection, intraperitoneal (i.p.) injection, and/or intramuscular (i.m.) injection. One or more such routes can be employed. Parenteral administration can be, for example, by bolus injection or by gradual perfusion over time. Alternatively or in addition, administration can be by the oral route.

In some embodiments, an injection is an intradermal (i.d.) injection. The compositions are in some embodiments suitable for administration of the antagonist and antigen by any acceptable route such as but not limited to oral (enteral), nasal, ophthal, and transdermal either separately or together. In some embodiments, the administration is subcutaneous, and in some embodiments the subcutaneous administration is by an infusion pump.

### V.B. Formulations

Pharmaceutical carriers, diluents, and excipients are generally added to the compositions or kits that are compatible with the active ingredients and acceptable for pharmaceutical use. Examples of such carriers include but are not limited to water, saline solutions, dextrose, and/or glycerol. Combinations of carriers can also be used.

The compositions of the presently disclosed subject matter can further incorporate additional substances to stabilize pH and/or to function as adjuvants, wetting agents, and/or emulsifying agents, which can serve to improve the effectiveness of the vaccine.

The antagonist/antigen compositions may include one or more adjuvants such as for example: montanide ISA-51 (Seppic Inc., Fiarfield, N.J., United States of America); QS-21 (Aquila Biopharmaceuticals, Inc., Framingham, Mass., United States of America); Arlacel A; oeleic acid; tetanus helper peptides (such as but not limited to QYIKANSKFIGITEL (SEQ ID NO: 2376) and/or AQYIKANSKFIGITEL (SEQ ID NO: 2377); GM-CSF; cyclophosamide; bacillus Calmette-Guerin (BCG); Corynbacterium parvum; levamisole, azimezone; isoprinisone; dinitrochlorobenezene (DNCB); keyhole limpet hemocyanin (KLH); Freunds adjuvant (complete and incomplete); mineral gels; aluminum hydroxide (Alum); lysolecithin; pluronic polyols; polyanions; peptides; oil emulsions; nucleic acids (such as but not limited to souble-stranded RNAs; dsRNA) dinitrophenol; diphtheria toxin (DT); toll-like receptor (TLR; such as but not limited to TLR3, TLR4, TLR7, TLR8, and/or TLR9) agonists (including but not limited to endotoxins such as lipopolysaccharide (LPS); monophosphoryl lipid A (MPL); and/or polyinosinic-polycytidylic acid (poly-ICLC/HILTONOL.RTM.; Oncovir, Inc., Washington, D.C., United States of America); IMO-2055; glucopyranosyl lipid A (GLA); QS-21 (a saponin extracted from the bark of the Quillaja saponaria tree, also known as the soap bark tree or Soapbark); resiquimod (a TLR7/8 agonist); CDX-1401 (a fusion protein consisting of a fully human monoclonal antibody with specificity for the dendritic cell receptor DEC-205 linked to the NY-ESO-1 tumor antigen); Juvaris' Cationic Lipid-DNA Complex; Vaxfectin; and combinations thereof.

Polyinosinic-Polycytidylic acid (Poly IC) is a double-stranded RNA (dsRNA) that acts as a TLR3 agonist. To increase half-life, it has been stabilized with polylysine and carboxymethylcellulose as poly-ICLC. It has been used to induce interferon in cancer patients, with intravenous doses up to 300 μg/kg. Like poly-IC, poly-ICLC is a TLR3 agonist. TLR3 is expressed in the early endosome of myeloid DC; thus poly-ICLC preferentially activates myeloid dendritic cells, thus favoring a Th1 cytotoxic T cell response. Poly-ICLC activates natural killer (NK) cells, induces cytolytic potential, and induces IFN.gamma. from myeloid DC.

In some embodiments, an adjuvant is provided at about or at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg per dose or per kg in each dose. In some embodiments, the adjuvant is provided in a dosage of at least or about 0.1, 0.2, 0.3, 0.40, 0.50, 0.60, 0.70, 0.80, 0.90, 0.100, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90, 5.00, 5.10, 5.20, 5.30, 5.40, 5.50, 5.60, 5.70, 5.80, 5.90, 6.00, 6.10, 6.20, 6.30, 6.40, 6.50, 6.60, 6.70, 6.80, 6.90, 7.00, 7.10, 7.20, 7.30, 7.40, 7.50, 7.60, 7.70, 7.80, 7.90, 8.00, 8.10, 8.20, 8.30, 8.40, 8.50, 8.60, 8.70, 8.80, 8.90, 9.00, 9.10, 9.20, 9.30, 9.40, 9.50, 9.60, 9.70, 9.80, 9.90, or 10.00 grams per dose or per kg in each dose. In some embodiments, the adjuvant is given at about or at least 10, 15, 20, 25, 50, 75, 100, 125, 150, 175, 150, 175, 200, 225, 250, 275, 300, 325, 350, 375, 400, 425, 450, 500, 525, 550, 575, 600, 625, 675, 700, 725, 750, 775, 800, 900, 1000, 1100, 1200, 1300, 1400, 1500, 1600, 1700, 1800, 1900, or 2000 endotoxin units (“EU”) per dose. The compositions of the presently disclosed subject matter can in some embodiments be provided with an administration of cyclophosamide around the time (e.g., about or at least 1, 2, 3, or 4 weeks or days before and/or after) of the initial dose of the composition. Exemplary non-limiting doses of cyclophosamide are about or at least 100, 200, 300, 400, 500, 600, 700, 800, 900, or 1000 Mg/m²/day over about or at least 1, 2, 3, 4, 5, 6, 7, 8, 9, or 10 days.

The compositions can comprise the antagonist and/or antigen peptides or nucleic acids encoding the antagonist and/or antigen in the free form and/or in the form of a pharmaceutically acceptable salt. As used herein, “a pharmaceutically acceptable salt” refers to a derivative of the composition, which is modified by making acid or base salts of the agent. For example, acid salts are prepared from the free base (typically wherein the neutral form of the drug has a neutral —NH₂ group) involving reaction with a suitable acid. Suitable acids for preparing acid salts include both organic acids such as but not limited to acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids such as but not limited to hydrochloric acid, hydrobromic acid, sulfuric acid, nitric acid phosphoric acid, and the like. Conversely, basic salts of acid moieties that can be present on peptide or nucleic acids are in some embodiments prepared using a pharmaceutically acceptable base such as but not limited to sodium hydroxide, potassium hydroxide, ammonium hydroxide, calcium hydroxide, trimethylamine, and the like. By way of example and not limitation, the compositions can comprise peptides or nucleic acids as salts of acetic acid (acetates), ammonium, or hydrochloric acid (chlorides).

In some embodiments, a composition can include one or more sugars, sugar alcohols, amino acids such but not limited to glycine, arginine, glutamic acid, and/or others as framework formers. The sugars can be mono-, di-, or trisaccharides. These sugars can be used alone and/or in combination with sugar alcohols. Exemplary sugars include glucose, mannose, galactose, fructose, or sorbose as monosaccharides; sucrose, lactose, maltose, and trehalose as disaccharides; and raffinose as a trisaccharide. A sugar alcohol can be, for example, mannitose. In some embodiments, the composition comprises sucrose, lactose, maltose, trehalose, mannitol, and/or sorbitol. In some embodiments, the composition comprises mannitol.

Furthermore, in some embodiments compositions can include physiological well-tolerated excipients (see Handbook of Pharmaceutical Excipients, 5th ed., edited by Raymond Rowe, Paul Sheskey and Sian Owen, Pharmaceutical Press (2006)) such as antioxidants like ascorbic acid or glutathione; preserving agents such as phenole, m-cresole, methyl- or propylparabene, chlorobutanol, thiomersal, and/or benzalkoniumchloride; stabilizers, framework formers such as sucrose, lactose, maltose, trehalose, mannitose, mannitol, and/or sorbitol; mannitol and/or lactose and solubilizers such as polyethyleneglycols (PEG; e.g., PEG 3000, 3350, 4000, or 6000), cyclodextrines (e.g., hy droxypropyle-β-cyclodextrine, sulfobutylethyl-β-cyclodextrine, or γ-cyclodextrine), dextranes, or poloxaomers (e.g., poloxamer 407 or poloxamer 188); or TWEEN® 20 or TWEEN® 80. In some embodiments, one or more well-tolerated excipients can be included, optionally selected from the group consisting of antioxidants, framework formers, and stabilizers.

In some embodiments, the pH for intravenous and/or intramuscular administration is selected from pH 2 to pH 12. In some embodiments, the pH for subcutaneous administration is selected from pH 2.7 to pH 9.0 as the rate of in vivo dilution is reduced resulting in more potential for irradiation at the injection site (Strickley, 2004).

### V.C. Dosages

It is understood that a suitable dosage of an antagonist/antigen composition (which may be administered in the same or different formulations) can depend upon the age, sex, health, and/or weight of the recipient, the kind of concurrent treatment, if any, the frequency of treatment, and the nature of the effect desired. However, it is understood that dosages can be tailored to the individual subject, as determined by the researcher or clinician. The total dose required for any given treatment will in some embodiments be determined with respect to a standard reference dose based on the experience of the researcher or clinician, such dose being administered either in a single treatment or in a series of doses, the success of which will depend on the production of a desired immunological result (such as but not limited to successful production of a T helper cell and/or CTL-mediated response to the checkpoint antagonist/antigen immunogen composition, which response gives rise to the prevention and/or treatment desired).

In some respects, the antagonist/antigen composition may be delivered as one composition or as two or more independent compositions. The two or more independent compositions comprise one or more immune checkpoint antagonists and one or more antigens, which may be administered simultaneously, concurrently, or sequentially. It is further possible to administer such compositions with a delay between dosages of one composition relative to another.

Thus, in some embodiments the overall administration schedule is considered in determining the success of a course of treatment and not whether a single dose, given in isolation, would or would not produce the desired immunologically therapeutic result or effect. As such, a therapeutically effective amount (i.e., in some embodiments that amount that produces a desired T helper cell and/or CTL-mediated response) can depend on the antagonist/antigenic composition used, the nature of the disease condition, the severity of the disease condition, the extent of any need to prevent such a condition where it has not already been detected, the manner of administration dictated by the situation requiring such administration, the weight and state of health of the individual receiving such administration, and/or the sound judgment of the clinician or researcher. In some embodiments, the efficacy of administering additional doses and/or of increasing or decreasing the interval can be continually re-evaluated in view of the recipient's immunocompetence (including but not limited to the level of T helper cell and/or CTL activity with respect to tumor-associated or tumor-specific antigens).

The concentration of the T helper or CTL stimulatory antigenic peptides or nucleic acids of the presently disclosed subject matter in pharmaceutical formulations can be subject to wide variation, including anywhere from less than 0.01% by weight to as much as 50% or more. Factors such as volume and viscosity of the resulting composition can in some embodiments also be considered. The solvents or diluents used for such compositions can include water, phosphate buffered saline (PBS), and/or saline, or any other possible carriers or excipients.

The antagonist/antigen compositions or immunogens of the presently disclosed subject matter can in some embodiments also be contained in artificially created structures such as liposomes, which structures in some embodiments can contain additional molecules such as but not limited to proteins or polysaccharides, inserted in the outer membranes of said structures and having the effect of targeting the liposomes to particular areas of the body and/or to particular cells within a given organ or tissue. Such targeting molecules can in some embodiments comprise an immunoglobulin. Antibodies can work particularly well for targeting of liposomes and/or other scaffolds to tumor cells.

Single i.d., i.m., s.c., i.p., and/or i.v. doses of in some embodiments about 1 to 50 μg, in some embodiments about 1 to 100 μg, in some embodiments about 1 to 500 μg, in some embodiments about 1 to 1000 μg, in some embodiments about 1 to 50 mg, in some embodiments about 1 to 100 mg, in some embodiments about 1 to 500 mg, or in some embodiments about 1 to 1000 mg of antagonist or antigen peptide or nucleic acids thereof in the composition can be given and can depend from the respective compositions of the peptides or nucleic acids with respect to total amount for all peptides or nucleic acids in the composition or alternatively for each individual target peptide or nucleic acids in the composition. A single dose of a peptide or nucleic acids composition of the presently disclosed subject matter can in some embodiments have a peptide or nucleic acids amount (e.g., total amount for all peptides or nucleic acids in the composition or alternatively for each individual peptide or nucleic acids in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 μg. In some embodiments, a single dose of a peptide or nucleic acids composition of the presently disclosed subject matter can have a total peptide or nucleic acids amount (e.g., total amount for all peptides or nucleic acids in the composition or alternatively for each individual peptide or nucleic acids in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 mg. In some embodiments, the peptides or nucleic acids of a composition of the presently disclosed subject matter are present in equal amounts of about 100 micrograms per dose in combination with an adjuvant peptide or nucleic acids present in an amount of about 200 micrograms per dose.

In a single dose of the peptide or nucleic acids composition of the presently disclosed subject matter, the amount of each peptide or nucleic acids in the composition is in some embodiments equal or substantially equal. Alternatively, a ratio of the peptides or nucleic acids present in the least amount relative to the peptide or nucleic acids present in the greatest amount is about or at least 1:1.25, 1:1.5, 1:1.75, 1:2.0, 1:2.25, 1:2.5, 1:2.75, 1:3, 1:4, 1:5, 1:6, 1:7, 1:8, 1:9, 1:10, 1:20, 1:30; 1:40, 1:50, 1:100, 1:200, 1:500, 1:1000, 1:5000; 1:10,000; or 1:100,000. Alternatively, a ratio of the peptides or nucleic acids present in the least amount relative to the peptide or nucleic acids present in the greatest amount is about or at least 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15 to 20; 20 to 25; 1 to 40; 1 to 30; 1 to 20; 1 to 15; 10 to 40; 10 to 30; 10 to 20; 10 to 15; 20 to 40; 20 to 30; or 20 to 25; 1 to 100; 25 to 100; 50 to 100; 75 to 100; 25 to 75, 25 to 50, or 50 to 75; 25 to 40; 25 to 50; 30 to 50; 30 to 40; or 30 to 75.

Single dosages can be given to a patient or nucleic acids about or at least 1, 2, 3, 4, or 5 times per day. Single dosages can be given to a patient about or at least 1, 2, 3, 4, 5, 6, 7, 8, 9, 10, 11, 12, 13, 14, 15, 16, 18, 19, 20, 21, 22, 23, 24, 36, 48, 60, or 72 hours subsequent to a previous dose.

Single dosages can be given to a patient about or at least 1, 2, 3, 4, 5, 6, or 7 times per week, or every other, third, fourth, or fifth day. Single doses can also be given every week, every other week, or only during 1, 2, or 3 weeks per month. A course of treatment can in some embodiments last about or at least 1, 2, 3, 4, 5, 6, 7, 8, 9, 10, 11. or 12 months.

In some embodiments, the single dosages of the compositions of the presently disclosed subject matter can be provided to a patient in at least two phases: e.g., during an initial phase and then during a subsequent phase. An initial phase can be about or at least 1, 2, 3, 4, 5, or 6 weeks in length. The subsequent phase can last at least or about 1, 2, 3, 4, 5, 6, 7, or 8 times as long as the initial phase. The initial phase can be separated from the subsequent phase by about or at least 1, 2, 3, 4, 5, 6, 7, 8, 9, 10, 11, or 12 weeks or months.

The peptide or nucleic acids composition dosage during the subsequent phase can be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times greater than during the initial phase.

The peptide or nucleic acids composition dosage during the subsequent phase can be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times less than during the initial phase.

In some embodiments, the initial phase is about three weeks and the second phase is about 9 weeks. The peptide or nucleic acids compositions can be administered to the patient on or about days 1, 8, 15, 36, 57, and 78.

As used above, the dosages for “peptides or nucleic acids” are each independently the immune checkpoint antagonist or the antigen in compositions of the invention. Therefore, the total amount for all peptides will refer to that specific antagonist or specific antigen relative to the remaining components in the composition.

### V.D. Kits and Storage

In some embodiments, a kit is disclosed comprising (a) a container that contains at least checkpoint antagonist/antigen composition as described herein, in solution or in lyophilized form, preferably in the form of nucleic acids encoding the antagonist and antigen; (b) optionally, a second container containing a diluent or reconstituting solution for the lyophilized formulation; and (c) optionally, instructions for (i) use of the solution or (ii) reconstitution and/or use of the lyophilized formulation. The kit may further comprise one or more of (iii) a buffer, (iv) a diluent, (v) a filter, (vi) a needle, or (v) a syringe. In some embodiments, the container is selected from the group consisting of: a bottle, a vial, a syringe, a test tube, or a multi-use container. In some embodiments, the peptide or nucleic acids composition is lyophilized.

The kits can contain exactly, about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, or more antigen/antagonist-containing compositions. Each composition in the kit can be administered at the same time or at different times.

In some embodiments, the kits can comprise a lyophilized formulation of the presently disclosed compositions and/or vaccines in a suitable container and instructions for its reconstitution and/or use. Suitable containers include, for example, bottles, vials (e.g., dual chamber vials), syringes (such as dual chamber syringes), and test tubes. The container can be formed from a variety of materials such as glass or plastic. In some embodiments, the kit and/or the container contain(s) instructions on or associated therewith that indicate(s) directions for reconstitution and/or use of a lyophilized formulation. For example, the label can indicate that the lyophilized formulation is to be reconstituted to antigen concentrations as described herein. The label can further indicate that the formulation is useful or intended for subcutaneous administration. Lyophilized and liquid formulations are typically stored at −20° C. to −80° C.

The container holding the checkpoint antagonist/antigen composition(s) can be a multi-use vial, which in some embodiments allows for repeat administrations (e.g., from 2-6 or more administrations) of the reconstituted formulation. The kit can further comprise a second container comprising a suitable diluent (e.g., sodium bicarbonate solution).

The kit can further include other materials desirable from a commercial and/or user standpoint, including other buffers, diluents, filters, needles, syringes, and package inserts with or without instructions for use.

The kits can have a single container that contains the formulation of the peptide or nucleic acids compositions with or without other components (e.g., other compounds or compositions of these other compounds) or can have a distinct container for each component.

Additionally, the kits can include a formulation of the peptide or nucleic acids compositions and/or vaccines packaged for use in combination with the co-administration of a second compound (such as adjuvants including but not limited to imiquimod), a chemotherapeutic agent, a natural product, a hormone or antagonist, an anti-angiogenesis agent or inhibitor, an apoptosis-inducing agent or a chelator) or a composition thereof. One or more of the components of the kit can be pre-complexed or one or more components can be in a separate distinct container prior to administration to a patient. One or more of the components of the kit can be provided in one or more liquid solutions. In some embodiments, the liquid solution is an aqueous solution. In a further embodiment, the liquid solution is a sterile aqueous solution. One or more of the components of the kit can also be provided as solids, which in some embodiments can be converted into liquids by addition of suitable solvents, which in some embodiments can be provided in another distinct container.

The container of a therapeutic kit can be a vial, a test tube, a flask, a bottle, a syringe, or any other structure suitable for enclosing a solid or liquid. Typically, when there is more than one component, the kit contains a second vial or other container that allows for separate dosing. The kit can also contain another container for a pharmaceutically acceptable liquid. In some embodiments, a therapeutic kit contains an apparatus (e.g., one or more needles, syringes, eye droppers, pipette, etc.), which enables administration of the agents of the disclosure that are components of the kit.

### V.E. Markers for Efficacy

When administered to a patient, the compositions of the presently disclosed subject matter are in some embodiments envisioned to have certain physiological effects including but not limited to the induction of a T cell mediated immune response.

### V.E.1. Immunohistochemistry, Immunofluorescence, Western Blots, Flow Cytometry

Commercially available antibodies for use in immunohistochemistry (IHC), immunofluorescence (IF), flow cytometry (FC), and/or western bloting (WB) can be used to characterize the cellular and molecular features of neogenesis. In some embodiments, such techniques can be employed to assay patient samples including but not limited to formalin-fixed, paraffin-embedded tissue samples for the presence or absence of and/or for a level of expression of one or more of CD1a, S100, CD83, DC-LAMP, CD3, CD4, CD8, CD20, CD45, CD79a, PNAd, TNF.alpha., LIGHT, CCL19, CCL21, CXCL12, TLR4, TLR7, FoxP3, PD-1, and Ki67 gene products. In some embodiments, flow cytometry is used to determine an expression level for one or more of CD3, CD4, CD8, CD13, CD14, CD16, CD19, CD45RA, CD45RO, CD56, CD62L, CD27, CD28, CCR7, FoxP3 (intracellular), and MHC-peptide tetramers for I MHC associated (phospho)-peptides. In some embodiments, a positive control is employed, which in some embodiments can comprise a tissue sample comprising normal human peripheral blood lymphocytes (PBL), PBL activated with CD3/CD28 beads (activated PBL), human lymph node tissue from non-melanoma patients (LN), and/or inflamed human tissue from a surgical specimen of Crohn's disease (Crohn's), although any other positive control cell and/or tissue can be employed.

### V.E.2 ELISpot Assay

In some embodiments, vaccination site infiltrating lymphocytes and lymphocytes from the sentinel immunized node (SIN) and vaccine site can be evaluated by ELISpot. ELISpot permits the direct counting of T cells reacting to antigen by production of INF.gamma.. Peripheral blood lymphocytes can be evaluated by ELISpot assay for the number of peptide-reactive T cells. Vaccine site infiltrating lymphocytes and SIN lymphocytes can be compared to those in peripheral blood. It is envisioned that positive results of the ELISpot assay correlates with increased patient progression free survival. Progression free survival is defined as the time from start of treatment until death from any cause or date of last follow up.

### V.E.3 Tetramer Assay

Peripheral blood lymphocytes and lymphocytes from the SIN and vaccine site can be evaluated by flow cytometry after incubation with MHC-peptide tetramers for the number of peptide-reactive T cells.

### V.E.4 Proliferation Assay/Cytokine Analysis

Peripheral blood mononuclear cells (PBMC), vaccine-site inflammatory cells, and/or lymphocytes from the SIN isolated from subjects can be evaluated for CD4⁺ T cell reactivity to, e.g., tetanus helper peptide mixture, using a ³H-thymidine uptake assay. Additionally, Th1 (IL-2, IFNγ, TNFα), Th2 (IL-4, IL-5, IL-10), Th17 (IL-17, and IL23), and T-reg (TGF-β) cytokines in media from 48 hours in that proliferation assay can be used to determine if the microenvironment supports generation of Th1, Th2, Th17, and/or T-reg responses. In some embodiments, one or both of the following peptides are used as negative controls: a tetanus peptide and the PADRE peptide (aK(X)VAAWTLKAa; SEQ ID NO: 2378).

### V.E.5 Evaluation of Tumors

In some embodiments, tumor tissue collected prior to treatment or at the time of progression can be evaluated by routine histology and immunohistochemistry. Alternatively or in addition, in vitro evaluations of tumor tissue and tumor infiltrating lymphocytes can be performed.

### V.E.6 Studies of Homing Receptor Expression

Patient samples can be studied for T cell homing receptors induced by vaccination with the compositions of the presently disclosed subject matter. These include, but are not limited to, integrins (including but not limited to αEβ7, α1β1, α4β1), chemokine receptors (including but not limited to CXCR3), and selectin ligands (including but not limited to CLA and PSL) on lymphocytes, and their ligands in the vaccine sites and SIN. In some embodiments, these can be assayed by immunohistochemistry, flow cytometry, and/or any other appropriate technique(s).

### V.E.7 Studies of Gene and Protein Expression

Differences in gene expression and/or differences in protein expression profiles can be determined by high-throughput screening assays (e.g., nucleic acid chips, protein arrays, etc.) of samples isolated from vaccine sites and/or SIN.

### VI. Antibodies and Antibody-Like Molecules

Antibodies and antibody-like molecules (including but not limited to T cell receptors) specific for antigen peptides and/or target peptide/MHC complexes are in some embodiments useful for analyzing biological samples. In some embodiments, an analysis can comprise determining the pathological nature of tumor margins.

Antibodies and antibody-like molecules can also be used in the composition as a checkpoint antagonists. In some embodiments, such molecules can be used as therapeutics that target cells, including but not limited to tumor cells.

As used herein, the terms “antibody”, “antibody peptide(s)”, and “antibody-like molecule(s)” refer to an intact antibody, a binding fragment thereof (i.e., a fragment of an antibody that comprises a paratope), or a polypeptide that can specifically recognize an antigen or epitope and bind to the same in a fashion that mimics antibody binding. In some embodiments, antibodies, antibody peptides, and antibody-like molecules compete with intact antibodies for specific binding to an antigen or epitope.

In some embodiments, antibody fragments can be produced by recombinant DNA techniques and/or by enzymatic and/or chemical cleavage of intact antibodies. Antibody fragments thus include but are not limited to Fab fragments, Fab′ fragments, F(ab′)2 fragments, Fv, and single-chain antibodies including but not limited to single-chain fragment variable (scFv) antibodies. An antibody is said to be “monospecific” if each of its paratopes is identical and/or binds to the same epitope. Similarly, “bispecific” or “bifunctional” antibodies comprise paratopes that bind to different antigens and/or epitopes. In some embodiments, an antibody substantially inhibits adhesion of a receptor to a counterreceptor when an excess of antibody reduces the quantity of receptor bound to counterreceptor by at least about 20%, 40%, 60%, 80%, 85%, 90%, 95%, or more as measured by, for example, an in vitro competitive binding assay.

The term “MHC” as used herein refer to the Major Histocompability Complex, which is defined as a set of gene loci specifying major histocompatibility antigens. The term “HLA” as used herein will be understood to refer to Human Leukocyte Antigens, which is defined as the histocompatibility antigens found in humans. As used herein, “HLA” is the human form of “MHC”. IN murine species, the MHC is referred to as the “H-2” complex.

The terms “MHC light chain” and “MHC heavy chain” as used herein refer to particular portions of a MHC molecule. Structurally, class I molecules are heterodimers comprised of two noncovalently bound polypeptide chains, a larger “heavy” chain (.alpha.) and a smaller “light” chain (β2-microglobulin or .beta.2m). The polymorphic, polygenic heavy chain (45 kDa), encoded within the MHC on chromosome human 6 is subdivided into three extracellular domains (designated 1, 2, and 3), one intracellular domain, and one transmembrane domain. The two outermost extracellular domains, 1 and 2, together form the groove that binds to antigenic peptides and/or other epitopes. Thus, interaction with the TCR occurs at this region of the protein. Domain 3 of the molecule contains the recognition site for the CD8 protein on the CTL. This interaction serves to stabilize the contact between the T cell and an antigen-presenting cell (APC). The invariant light chain (12 kDa), encoded on human chromosome 15, consists of a single, extracellular polypeptide. The terms “MHC light chain”, “β2-microglobulin”, and “β2m” are used interchangeably herein.

The term “epitope” includes any protein determinant capable of specific binding to an antibody, antibody peptide, and/or antibody-like molecule (including but not limited to a T cell receptor) as defined herein. Epitopic determinants typically consist of chemically active surface groups of molecules such as amino acids or sugar side chains and generally have specific three dimensional structural characteristics as well as specific charge characteristics. An antibody or antibody-like molecule is said to “specifically” bind an antigen when the dissociation constant (K_{d}) is in some embodiments less than about 1 μM, in some embodiments less that about 100 nM, and in some embodiments less than about 10 nM. Interactions between antibodies and antibody-like molecules and an eptiope can also be characterized by an affinity constant (Kₐ). In some embodiments, a Kₐ of less than about 10⁷/M is considered “high affinity”.

The term “antibody” is used in the broadest sense, and covers monoclonal antibodies (including full length monoclonal antibodies), polyclonal antibodies, multispecific antibodies (e.g., bispecific and/or trispecific antibodies), and antibody fragments (including but not limited to Fab, F(ab′)2 and Fv fragments) as well as antibody-like molecules provided that they exhibit the desired biological activity (e.g., antigen binding). Antibodies (Abs) and immunoglobulins (Igs) are glycoproteins that in some embodiments have the same structural characteristics. The term is also meant to encompass “antibody like molecules” and other members of the immunoglobulin superfamily including, but not limited to T cell receptors, MHC molecules, and other polypeptides that contain one or more antigen-binding regions and/or variable regions including, but not limited to complementary determining regions (CDRs) that specifically bind the target peptides disclosed herein.

The term “antibody” also encompasses soluble T cell receptor (TCR) cytoplasmic domains that are stable at low concentrations and which can recognize MHC-peptide complexes. See e.g., U.S. Patent Application Publication No. 2002/0119149, which is incorporated by reference. Such soluble TCRs can in some embodiments be conjugated to immunostimulatory peptides and/or proteins, and/or moieties such as but not limited to CD3 agonists (e.g., anti-CD3 antibodies). The CD3 antigen is present on mature human T cells, thymocytes, and a subset of natural killer cells. It is associated with the TCR and is involved in signal transduction of the TCR. Antibodies specific for the human CD3 antigen are well known. One such antibody is the murine monoclonal antibody OKT3 which was the first monoclonal antibody approved by the FDA. OKT3 is reported to be a potent T cell mitogen (Van Wauve, 1980; U.S. Pat. No. 4,361,539) and a potent T cell killer (Wong, 1990). Other antibodies specific for the CD3 antigen have also been reported (see PCT International Patent Application Publication No. WO 2004/106380; U.S. Patent Application Publication No. 2004/0202657; U.S. Pat. Nos. 6,750,325; 6,706,265; Great Britain Patent Publication GB 2249310A; Clark et al., 1989; U.S. Pat. No. 5,968,509; U.S. Patent Application Publication No. 2009/0117102). Immune mobilising mTCR Against Cancer (ImmTAC; Immunocore Limited, Milton Partk, Abington, Oxon, United Kingdom) are bifunctional proteins that combine affinity monoclonal T cell receptor (mTCR) targeting with a therapeutic mechanism of action (i.e., an anti-CD3 scFv).

Native antibodies and immunoglobulins are generally heterotetrameric glycoproteins of about 150,000 daltons (Da) composed of two identical light (L) chains and two identical heavy (H) chains. Each light chain is linked to a heavy chain by a covalent disulfide bond. Disulfide bonds also link the heavy chains of intact antibodies, although the number of disulfide bonds between the heavy chains of different immunoglobulin isotypes can vary. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., 1985; Novotny & Haber, 1985).

An “isolated” antibody is one which has been identified and/or separated and/or recovered from a component of the environment in which it was produced or otherwise present. Contaminant components of its production environment are materials that in some embodiments interfere with diagnostic and/or therapeutic uses for the antibody, and in some embodiments can include enzymes, hormones, and other proteinaceous or nonproteinaceous solutes. In some embodiments, an antibody can be purified as measurable by one or more of the following methods: 1) to greater than 50%, 75%, 80%, 85%, 90%, 95%, 96%, 97%, 98%, or 99% by weight of antibody as determined by the Lowry method; 2) to a degree sufficient to obtain at least 10 or 15 residues of N-terminal or internal amino acid sequence by use of a spinning cup sequentator; or 3) to homogeneity by SDS-PAGE under reducing or non-reducing conditions using Coomasie blue or, in some embodiments, silver stain. Isolated antibodies include an antibody in situ within recombinant cells since at least one component of the antibody's natural environment will not be present. Ordinarily, however, isolated antibodies will be prepared by a method that comprises at least one purification step.

The terms “antibody mutant” and “antibody variant” refer to antibodies that relative to a reference antibody comprise one or more amino acid sequence differences, wherein one or more of the amino acid residues have been modified such as but not limited to substitution and/or deletion. Such mutants and/or variants comprise in some embodiments less than 100%, 99%, 98%, 97%, 96%, 95%, 90%, 85%, 80%, or 75% sequence identity and/or similarity to the amino acid sequence of either the heavy or light chain variable domain amino acid sequence of the reference antibody.

The term “variable” in the context of variable domain of antibodies, refers to the fact that certain portions of the variable domains differ extensively in sequence among antibodies and are used in the binding and specificity of each particular antibody for its particular antigen. However, sequence variability is generally not evenly distributed throughout the variable domains of antibodies. Typically, seqeunce variability is concentrated in three segments called complementarity determining regions (CDRs; also known as hypervariable regions) both in the light chain and heavy chain variable domains.

There are at least two techniques for determining CDRs: (1) an approach based on cross-species sequence variability (i.e., Kabat et al., 1991); and (2) an approach based on crystallographic studies of antigen-antibody complexes (Chothia et al., 1989). The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat et al., 1991) The constant domains are generally not involved directly in binding between antibody and antigen, but exhibit various effector functions such as but not limited to participation of the antibody in antibody-dependent cellular toxicity.

The term “antibody fragment” refers to a portion of a full-length antibody, generally the antigen binding or variable region. Examples of antibody fragments include Fab, Fab′, F(ab′)₂, and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). As used herein, the phrase “functional fragment” with respect to antibodies refers in some embodiments to a fragment that contains at least one antigen-binding domain (referred to as a “paratope”), and thus includes, but is not limited to Fv, F(ab) and F(ab′)₂ fragments.

An “Fv” fragment is the minimum antibody fragment which contains a complete antigen recognition and binding site. This region consists of a heterodimer of one heavy and one light chain variable domain in a tight, non-covalent or covalent association (V_{H}-V_{L} dimer). It is in this configuration that the three CDRs of each variable domain interact to define an antigen binding site (paratope) on the surface of the V_{H}-V_{L} dimer. Collectively, the six CDRs confer antigen binding specificity to the antibody. However, in some embodiments even a single variable domain (or half of an Fv comprising only three CDRs specific for an antigen) has the ability to recognize and bind antigen, although at a lower affinity than the entire binding site.

The Fab or F(ab) fragment also contains the constant domain of the light chain and the first constant domain (CHI) of the heavy chain. Fab′ fragments differ from Fab fragments by the addition of a few residues at the carboxyl terminus of the heavy chain CHI domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

The light chains of antibodies (immunoglobulin) from any vertebrate species can be assigned to one of two clearly distinct types, called kappa (κ) and lambda (λ), based on the amino sequences of the corresponding constant domain.

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses or isotypes (e.g., IgG₁, IgG₂, IgG₃, IgG₄, IgA₁, IgA₂, etc.). The heavy chains constant domains that correspond to the different classes of immunoglobulins are called alpha (α), delta (δ), epsilon (ε), gamma (γ), and mu (μ), respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, monoclonal antibodies can be advantageous in that they are typically synthesized from hybridomas and thus can be isolated in a form that is uncontaminated by other immunoglobulins. Methods for generating hybridomas are known in the art. The modifier “monoclonal” indicates the character of the antibody as being obtained from a substantially homogeneous population of antibodies, and is not to be construed as requiring production of the antibody by any particular method. By way of example and not limitation, monoclonal antibodies to be used in accordance with the presently disclosed subject matter can be made by the hybridoma method first described by Kohler & Milstein, 1975, or can be made by recombinant methods (see e.g., U.S. Pat. No. 4,816,567; Harlow & Lane, 1988). In some embodiments, the monoclonal antibodies for use with the presently disclosed subject matter can be isolated from phage antibody libraries using the techniques described in Clackson et al., 1991 and/or Marks et al., 1991.

Utilization of the monoclonal antibodies of the presently disclosed subject matter can in some embodiments comprise administering one or more monoclonal antibodies to a subject, such as but not limited to a human subject. However, when the monoclonal antibodies are produced in a non-human animal, such as a rodent, administration of such antibodies to a human patient can elicit an immune response, wherein the immune response is directed towards the administered antibodies themselves. Such reactions can limit the duration and effectiveness of such a therapy. In order to overcome such a problem, the monoclonal antibodies of the presently disclosed subject matter can in some embodiments be “humanized”, that is, the antibodies are engineered such that antigenic portions thereof are removed and like portions of a human antibody are substituted therefor, while the antibodies' affinity for specific peptide/MHC complexes is retained. This engineering can involve a few amino acids, or can include the entire framework regions of the antibody, leaving only the complementarity determining regions of the parent antibody intact. Several methods of humanizing antibodies are known in the art and are disclosed in U.S. Pat. Nos. 6,180,370; 6,054,927; 5,869,619; 5,861,155; 5,712,120; and 4,816,567, the entire disclosure of each of which is hereby expressly incorporated herein by reference in its entirety.

Humanized forms of antibodies are thus chimeric immunoglobulins, immunoglobulin chains, or fragments thereof (such as Fv, Fab, Fab′, F(ab)₂ or other antigen-binding subsequences of antibodies) that are principally comprised of the sequence of a human immunoglobulin, but that contain at least some subsequences derived from a non-human immunoglobulin. Humanization can be performed following the method of Winter and co-workers by substituting rodent CDRs or CDR sequences for the corresponding sequences of a human antibody (Jones et al., 1986; Riechmann et al., 1988; Verhoeyen et al., 1988; see also U.S. Pat. No. 5,225,539). In some embodiments, Fv framework residues of a human immunoglobulin are replaced with corresponding non-human residues from an antibody of interest. Humanized antibodies can also comprise residues which are found neither in the recipient antibody nor in the imported CDR or framework sequences. In general, the humanized antibody comprises substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally can also comprise at least a portion of an immunoglobulin constant region (Fc), typically that of a human immunoglobulin (Jones et al., 1986; Riechmann et al., 1988; and Presta, 1992).

Exemplary publications relating to the generation and/or use of humanized antibodies include Sandborn et al., 2001; Mihara et al., 2001; Yenari et al., 2001; Morales et al., 2000; Richards et al., 1999; Yenari et al., 1998; and Shinkura et al., 1998; each of which is expressly incorporated by reference herein in its entirety. For example, a treatment protocol that can be utilized in such a method includes a single dose, generally administered intravenously, of 10-20 mg of humanized mAb per kg (see e.g., Sandborn et al., 2001). In some cases, alternative dosing patterns can be appropriate, such as the use of three infusions, administered once every two weeks, of 800-1600 mg or even higher amounts of humanized mAb (see e.g., Richards et al., 1999). However, it is to be understood that the presently disclosed subject matter is not limited to the treatment protocols described herein, and further that other treatment protocols that are known to one of ordinary skill in the art can be employed in the methods of the presently disclosed subject matter.

In some embodiments, the presently disclosed subject matter further relates to fully human monoclonal antibodies against specific target peptide/MHC complexes. Fully human antibodies essentially relate to antibody molecules in which the entire sequence of both the light chain and the heavy chain, including the CDRs, arise from human genes. Such antibodies are referred to herein as “human antibodies” or “fully human antibodies”.

Human monoclonal antibodies can be prepared by the trioma technique (see U.S. Pat. No. 4,714,681; PCT International Patent Application Publication No. WO 1999/047929); the human B-cell hybridoma technique (see Kozbor et al., 1983), and/or the EBV hybridoma technique (see Cole et al., 1985). In some embodiments, human monoclonal antibodies can be utilized in the practice of the presently disclosed subject matter and can be produced by using human hybridomas (see Cote et al., 1983) or by transforming human B-cells with Epstein Barr Virus in vitro (see Cole et al., 1985). In addition, human antibodies can also be produced using additional techniques, such as but not limited to phage display libraries (Hoogenboom et al., 1991; Marks et al., 1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., 1992; Lonberg et al., 1994; Fishwild et al., 1996; Neuberger, 1996; and Lonberg & Huszar, 1995.

Human antibodies can additionally be produced using transgenic non-human animals that have been modified to produce fully human antibodies in addition to or rather than the non-human animal's endogenous antibodies in response to challenge by an antigen. See PCT International Patent Application Publication No. WO 1994/02602. In some embodiments, endogenous genes encoding the heavy and light immunoglobulin chains present in the non-human animal have been deleted or otherwise inactivated, and nucleic acids encoding human heavy and light chain immunoglobulins have been inserted into the host's genome. The human genes are incorporated, for example, using yeast artificial chromosomes containing the requisite human DNA segments. An animal that provides all the desired modifications is then obtained as progeny by crossbreeding intermediate transgenic animals containing fewer than the full complement of the modifications.

One embodiment of such a non-human animal is a mouse termed the XENOMOUSE™, which is described in PCT International Patent Application Publication Nos. WO 1996/33735 and WO 1996/34096. The XENOMOUSE™ produces B cells that secrete fully human immunoglobulins. The antibodies can be obtained directly from the animal after immunization with an immunogen of interest, as, for example, a preparation of polyclonal antibodies, or alternatively from immortalized B cells derived from an immunized animal, such as hybridomas producing monoclonal antibodies. Additionally, the genes encoding the immunoglobulins with human variable regions can be recovered and expressed to obtain the antibodies directly and/or can be further modified to obtain analogs of antibodies such as, for example, single chain Fv molecules.

An example of a method for producing a non-human animal such as but not limited to a mouse that lacks expression of an endogenous immunoglobulin heavy chain is disclosed in U.S. Pat. No. 5,939,598, incorporated herein by reference. Such a non-human animal can be obtained by a method that comprises deleting the J segment genes from at least one endogenous heavy chain locus in an embryonic stem cell, thereby preventing rearrangement of the locus and formation of an RNA encoding a rearranged immunoglobulin heavy chain locus. In some embodiments, the deletion can be effected by a targeting vector that contains a selectable marker, Thereafter, a transgenic animal (e.g., a mouse) having somatic and germ cells containing the gene encoding the selectable marker can be produced from the embryonic stem cell. The transgenic animal would be expected to be unable to rearrange its endogenous immunoglobulin heavy chain locus, and thus would be expected to be unable to produce endogenous immunoglobulins.

A method for producing an antibody of interest, such as a human antibody, is also disclosed in U.S. Pat. No. 5,916,771, incorporated herein by reference. It includes introducing a first expression vector that contains a nucleotide sequence encoding a heavy chain into one mammalian host cell in culture, introducing a second expression vector containing a nucleotide sequence encoding a light chain into another mammalian host cell, and fusing the two cells to form a hybrid cell. The hybrid cell can express thus an antibody made up of a heavy chain and a light chain encoded by the first and second expression vectors.

Checkpoint antagonists and antigens disclosed herein are in some embodiments expressed on a variety of diseases, including various cancer cell types, autoimmune diseases, neurodegenerative, and infectious diseases, etc.. Thus, in some embodiments antibodies and antibody-like molecules against checkpoint-related peptides can be expressed from a nucleic acid vector as a checkpoint antagonist, and in combination with tumor associated antigen for treating, diagnosing, vaccinating, preventing, retarding, and attenuating a cancer such as but not limited to melanoma, ovarian cancer, breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer. Alternatively, the composition may include antagonists that are known antibodies used in combination with antigens of the present invention.

Antibodies generated with specificity for an antigen as disclosed herein can be used to detect the corresponding target peptides in a biological sample. The biological sample is in some embodiments isolated from an individual who is suspected of having cancer, and thus detection could serve to diagnose the cancer. Alternatively, the biological sample could be isolated from an individual known to have cancer, and detection of a target peptide therein can serve as an indicator of disease prognosis, cancer characterization, treatment efficacy, disease progression, or any combination thereof. Immunoassays that can be employed for these purposes are known in the art and include, but are not limited to, immunohistochemistry, flow cytometry, radioimmunoassay, western blotting, and ELISA. Biological samples suitable for such testing include, but are not limited to, cells, tissue biopsy specimens, whole blood, plasma, serum, sputum, cerebrospinal fluid, pleural fluid, and urine.

Kits can be prepared to assist in diagnosis, monitoring, and/or prognosis of diseases. In some embodiments, the kits facilitate the detection and/or measurement of cancer-specific phosphopeptides and/or phosphoproteins. Such kits can contain, in a single or divided container, a molecule comprising an antigen-binding region. In some embodiments, such molecules are antibodies or antibody-like molecules. Additional components that can be included in the kit include one or more of solid supports, detection reagents, secondary antibodies, instructions for use, vessels for running assays, gels, control samples, and the like. In some embodiments, an antibody or antibody-like molecules can optionally be directly or indirectly labeled.

Alternatively, the antibody or antibody-like molecules specific for targeted immune checkpoint-associated proteins and antigens can be conjugated to therapeutic agents. Exemplary therapeutic agents include, but are not limited to the following:

Alkylating Agents: Alkylating agents are drugs that directly interact with genomic DNA to prevent cells from proliferating. This category of chemotherapeutic drugs represents agents that affect all phases of the cell cycle (i.e., they are not cell cycle phase-specific). Alkylating agents include, but are not limited to nitrogen mustards, ethylenimenes, methylmelamines, alkyl sulfonates, nitrosoureas, and triazines. Particularly exemplary alkylating agents include but are not limited to busulfan, chlorambucil, cisplatin, cyclophosphamide (cytoxan), dacarbazine, ifosfamide, mechlorethamine (mustargen), and melphalan.

Antimetabolites: Antimetabolites disrupt DNA and RNA synthesis. Unlike alkylating agents, they specifically influence the cell cycle during S phase. Antimetabolites can be differentiated into various categories, such as folic acid analogs, pyrimidine analogs, purine analogs, and related inhibitory compounds. Antimetabolites include but are not limited to 5-fluorouracil (5-FU), cytarabine (Ara-C), fludarabine, gemcitabine, and methotrexate.

Natural Products: Natural products generally refer to compounds originally isolated from a natural source and identified as having a desirable pharmacological activity. Such compounds, including analogs and derivatives thereof, can be isolated from a natural source, chemically synthesized, and/or recombinantly produced by any technique known to those of skill in the art. Natural products include such categories as mitotic inhibitors, antitumor antibiotics, enzymes, and biological response modifiers.

Mitotic inhibitors include plant alkaloids and other natural agents that can in some embodiments inhibit protein synthesis required for cell division and in some embodiments inhibiting mitosis. They typically operate during a specific phase of the cell cycle. Mitotic inhibitors include, for example, docetaxel, etoposide (VP16), teniposide, paclitaxel, taxol, vinblastine, vincristine, and vinorelbine, amoung others.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. Exemplary vinca alkaloids include vinblastine (VLB) and vincristine.

Antibiotics: Certain antibiotics have both antimicrobial and/or cytotoxic activity. These drugs also interfere with DNA by chemically inhibiting enzymes and mitosis or altering cellular membranes. These agents are typically not cell cycle phase-specific. Examples of cytotoxic antibiotics include but are not limited to bleomycin, dactinomycin, daunorubicin, doxorubicin (Adriamycin), plicamycin (mithramycin), and idarubicin.

Miscellaneous Agents: Miscellaneous cytotoxic agents that do not fall into the previous categories include but are not limited to platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are non-limiting and it is contemplated that any known cytotoxic, cytostatic, and/or cytocidal agent can be attached to a targeting peptide of the presently disclosed subject matter and administered to a targeted organ, tissue, and/or cell type.

Chemotherapeutic (cytotoxic) agents including, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raioxifene, tamoxifen, taxol, temazolomide (an aqueous form of DTIC), transplatinum, vinblastine, methotrexate, vincristine, and any analogs and/or derivatives or variants of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog, derivative, or variant thereof.

The above disclosure generally describes the presently disclosed subject matter. A more complete understanding can be obtained by reference to the following specific examples which are provided herein for purposes of illustration only, and are not intended to limit the scope of the presently disclosed subject matter.

### EXAMPLES

The following Examples provide further illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Example 1

A plasmid is designed and constructed with one or more guide RNAs targeting human PDL1, the CRISPR-Cas9 protein, and antigen protein beta-amyloid having the sequences in the following Table 2:

TABLE 2

| Element | Sequence | SEQ ID NO: |
|---|---|---|
| Truncated PDL1 | CAGTTCTGCGCAGCTTCCCGAGG | 60 |
| gRNA | CTCGGGAAGCTGCGCAGAACTGG | |
| | TCGGGAAGCTGCGCAGAACTGGG | |
| CTLA4 gRNA | CACATGTGTAATACATATCTGGG | 61 |
| | GCACATGTGTAATACATATCTGG | |
| | TACACATGTGCACACACAGAAGG | |
| Truncated PDL1 | Atgaggatat ttgctgtctt tatattcatg acctactggc | 62 |
| (with deleted | atttgctgaa cgcatttact | |
| transmembrane | gtcacggttc ccaaggacct atatgtggta gagtatggta | |
| domain) | gcaatatgac aattgaatgc | |
| | aaattcccag tagaaaaaca attagacctg gctgcactaa | |
| | ttgtctattg ggaaatggag | |
| | gataagaaca ttattcaatt tgtgcatgga gaggaagacc | |
| | tgaaggttca gcatagtagc | |
| | tacagacaga gggcccggct gttgaaggac | |
| | cagctctccc tgggaaatgc tgcacttcag | |
| | atcacagatg tgaaattgca ggatgcaggg gtgtaccgct | |
| | gcatgatcag ctatggtggt | |
| | gccgactaca agcgaattac tgtgaaagtc aatgccccat | |
| | acaacaaaat caaccaaaga | |
| | attttggttg tggatccagt cacctctgaa catgaactga | |
| | catgtcaggc tgagggctac | |
| | cccaaggccg aagtcatctg gacaagcagt | |
| | gaccatcaag tcctgagtgg taagaccacc | |
| | accaccaatt ccaagagaga ggagaagctt ttcaatgtga | |
| | ccagcacact gagaatcaac | |
| | acaacaacta atgagatttt ctactgcact tttaggagat | |
| | tagatcctga ggaaaaccat | |
| | acagctgaat tggtcatccc agaactacct ctggcacatc | |
| | ctccaaatga [symbol] | |
| | [symbol] [symbol] [symbol] [symbol] | |
| | [symbol] [symbol] | |
| | ttaagaaaag ggagaatgat ggatgtgaaa | |
| | aaatgtggca tccaagatac aaactcaaag | |
| | aagcaaagtg atacacattt ggaggagacg taatccagca | |
| | ttggaacttc tgatcttcaa | |
| | gcagggattc tcaacctgtg gtt | |
| Her2/Neu | HER-2 kinase domain mutations, e.g., | |
| | G309A, D769H, D769Y, V777L, P780ins, | |
| | V842I and R896C | |
| Beta-amyloid | DAEFRHD SGYEVHHQKLVFF AEDVGS | 64 |
| | NKGAIIGLMVGGVVIA | |
| CRISPR-Cas9 | E.g., UniProtKB-Q99ZW2 (CAS9-STRP2) | |

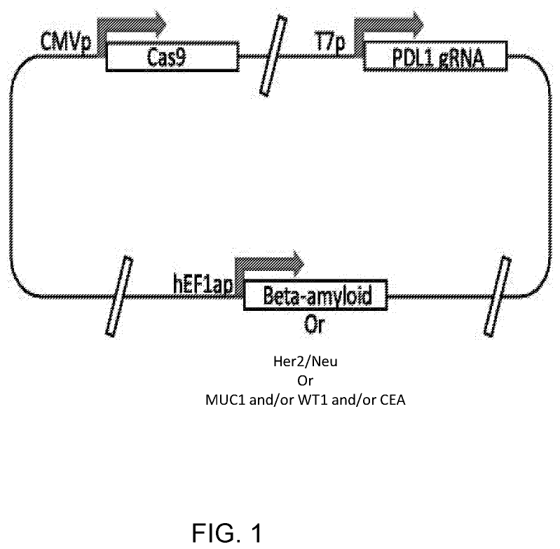

The CRISPR-Cas9 protein is expressed under a CMV promoter, a guide RNA is expressed from T7 promoter, and the beta-amyloid is expressed under the hEFla promoter, as depicted in FIG. 1. While FIG. 1 shows a single plasmid construction, it is within the scope of the invention to have these elements expressed in multiple DNA plasmids. This plasmid is delivered to a patient suffering from, for example, Alzheimer's disease. The current vector can be chemically synthesized based on publicly available sequence information.

### Example 2

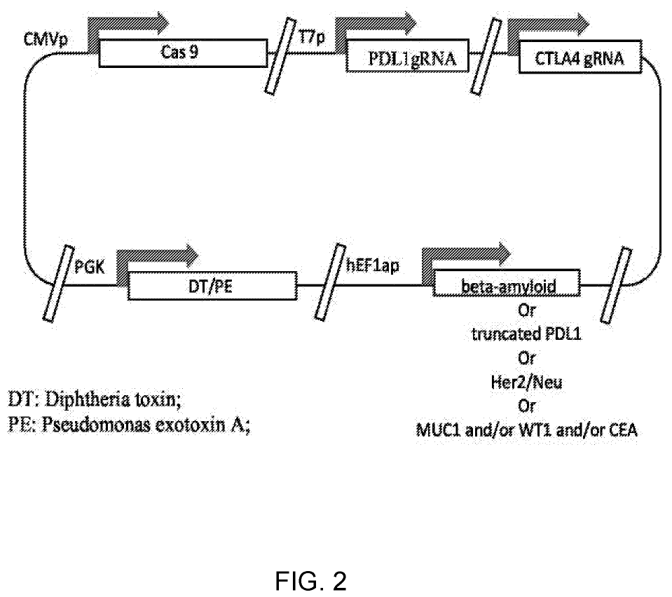

A single plasmid is constructed as described above in Example 1 using a guide RNA targeting PDL1 and/or CTLA4 (Table 1 above), the CRISPR-Cas9 protein, and Her2/Neu. FIG. 2 depicts a single plasmid, in which CRISPR-Cas9 protein is expressed from CMV promoter, guide RNA is expressed from T7 promoter, and the Her2/Neu is expressed from h-EFla promoter. Such composition can be used for treatment of diseases such as, but not limited to, breast cancer or other Her2/Neu expression cancer.

### Example 3

A single plasmid is constructed as described above in Example 1 using a guide RNA targeting PDL1 and/or CTLA4 (Table 1 above), the CRISPR-Cas9 protein, and Her2/Neu. FIG. 2 depicts a single plasmid, in which CRISPR-Cas9 protein is expressed from CMV promoter, PDL1 guide RNA is expressed from T7 promoter, CTLA4 gRNA is expressed under a promoter is expressed, and the Her2/Neu is expressed from h-EFla promoter.

### Example 4

A single plasmid is constructed as described above in Example 1 using an expression unit coding for cytotoxic protein (such as Pseudomonas Exotoxin A or Diphtheria toxin) under the PGK promoter, as shown in FIG. 2. Such configuration serve as safety guard to prevent the APCs with immune checkpoint knockout from continuously presenting self antigens to cause autoimmune reactions.

### Example 5

FIG. 2 depicts the use of multiple guide RNA (against same or different immune checking point inhibitors) that may be transcribed (FIG. 2) from same or different promoters. In FIG. 2, the PDL1 gRNA and the CTLA4 gRNA are both under the control of the T7p promoter. In the alternative, the use of different promoters are envisioned.

### Example 6

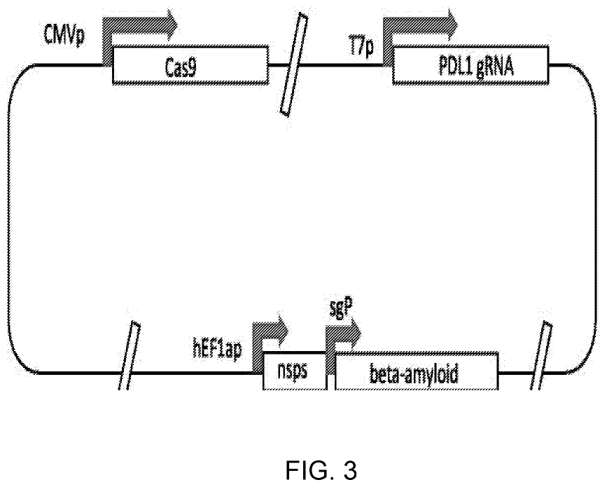

A single or multiple DNA vectors containing an alphavirus replicon is constructed. In this configuration, the guide RNA targeting PDL1 and/or CTLA4 sequence (Table 1), CRISPR-Cas9 protein, and beta-amyloid are expressed from single or multiple DNA vectors containing alphavirus replicon. In this case, FIG. 3 depicts that CRISPR-Cas9 protein is expressed from CMV promoter, guide RNA is expressed from T7 promoter, and the beta-amyloid is expressed under the subgenomic promoter of an alphavirus replicon. The lytic alphavirus vector is preferably used to provide extra safety guide. Such composition can be used for treat diseases such as, but not limited to, Alzheimer's disease and cancers.

### Example 7

A single plasmid is constructed as described above in Example 1 the guide RNA targeting PDL1 and/or CTLA4 (Table 1), CRISPR-Cas9 protein, and truncated (transmembrane domain deletion) human or different species of PDL1 (Table 2) and are expressed from single DNA plasmid. FIGS. 1 and 2 depict that CRISPR-Cas9 protein is expressed under the CMV promoter, guide RNA is expressed under the T7 promoter, and the truncated PDL1 is expressed under the h-EFla promoter. Such composition can produce auto-antibody against PDL1, and can be used for treatment of diseases such as, but not limited to, bladder cancer, NSCLC, or may be used for autoimmune disease animal model generation.

### Example 8

As depicted in FIG. 3, the guide RNA targeting PDL1 and/or CTLA4 (Table 1, the guide RNA sequences are selected to have no complementary sequence with truncated PDL1 gene), CRISPR-Cas9 protein, and truncated (no transmembrane domain) PDL1 is expressed from a single or multiple DNA vectors containing alphavirus replicon. In this example, CRISPR-Cas9 protein is expressed from CMV promoter, guide RNA is expressed from T7 promoter, and the truncated PDL1 is expressed from subgenomic promoter of an alphavirus replicon. The lytic alphavirus vector is preferably used to provide extra safety guide. Such composition can be used for treat diseases such as, but not limited to, bladder cancer and NSCLC (non-small cell lung cancer). Alternatively, it may be used for autoimmune disease animal model generation.

### Example 9

As an alternative to the configuration set forth in Example 8, the guide RNA targeting PDL1 and/or CTLA4 sequence (Table 1), CRISPR-Cas9 protein, and Her2/Neu (Table 1) are expressed from a single or multiple DNA vectors containing alphavirus replicon. As shown in FIG. 3, CRISPR-Cas9 protein is expressed from CMV promoter, guide RNA is expressed from T7 promoter, and the Her2/Neu is expressed from subgenomic promoter of an alphavirus replicon. The lytic alphavirus vector is preferably used to provide extra safety guide. Such compositions can be used for treatment of diseases such as, but not limited to, breast cancer and other Her2/Neu expression cancer.

### Example 10

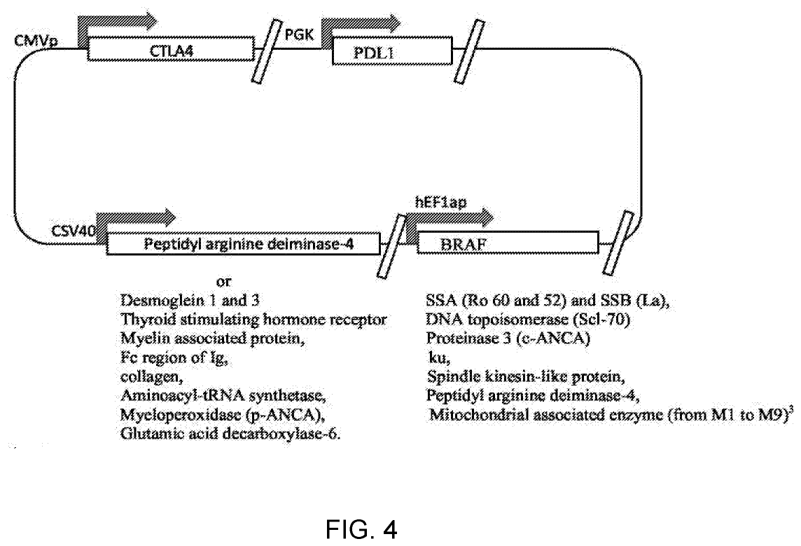

A DNA vector is constructed in which the PDL1 and/or CTLA4 protein under the control of the CMV promoter, PDL1 protein is under control of the PGK promoter, and BRAF is expressed under the hEFla promoter and/or peptidyl arginine deiminase-4 protein is expressed under the SV40 promoter. This vector is depicted in FIG. 4.

### Example 11

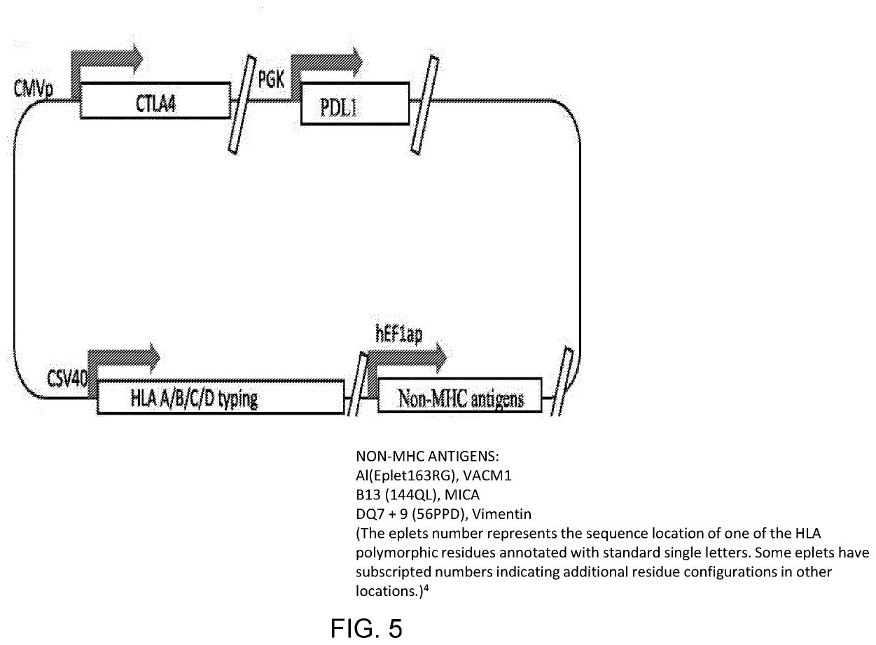

A vector is constructed as depicted in FIG. 5 comprising CTLA4 protein expressed under the CMV promoter, PDL1 antigen expressed under the PGK promoter, VACM1 expressed under the hEFla promoter and/or donor unmatched HLA DQ7+9 (56PPD) expressed under the SV40 promoter.

### REFERENCES

1. Ott P A, Hodi F S, Robert C. CTLA-4 and PD-1/PD-L1 blockade: new immunotherapeutic modalities with durable clinical benefit in melanoma patients. Clin Cancer Res. 2013;19(19):5300-9.
2. Sponaas A M, Moharrami N N, Feyzi E, Standal T, Holth Rustad E, Waage A, Sundan A. PDL1 Expression on Plasma and Dendritic Cells in Myeloma Bone Marrow Suggests Benefit of Targeted anti PD1-PDL1 Therapy. PLoS One. 2015;10(10):e0139867.
3. Racanelli V, Prete M, Musaraj G, Dammacco F, Perosa F. Autoantibodies to intracellular antigens generation and pathogenetic role. Autoimmunity Reviews 2011,10:503-508.
4. Duquesnoy R J, Reflections on HLA Epitope-Based Matching for

Transplantation. Frontiers in Immunology 2016; 7:1-8.
5. Gelao L, Criscitiello C, Esposito A, Goldhirsch A and Curigliano G. Immune Checkpoint Blockade in Cancer Treatment: A double-edged sword cross-targeting the host as an “innocent bystander”. Toxins 2014, 6, 914-933
6. PDL1 Expression on Plasma and Dendritic Cells in Myeloma Bone Marrow Suggests Benefit of Targeted anti PD1-PDL1 Therapy. Sponaas AM et al. PLoS One. 2015 Oct. 7; 10(10):e0139867.
7. Raya etal.,Targeting PD1PDL1 immune checkpoint in plasmacytoid dendritic cell inter actions with T cells, natural killer cells and multiple myeloma cells. Leukemia. 2015 June; 29(6):1441-4.
8. Weiskopf K, Weissman I L.,

Macrophages are critical effectors of antibody therapies for cancer.MAbs. 2015; 7(2):303-10.
9. Remy-Ziller C, et al. Sequential administration of MVA-based vaccines and PD-1/PD-L1-blocking antibodies confers measurable benefits on tumor growth and survival: preclinical studies with MVA-βGal and MVA-MUC1 (TG4010) in a murine tumor model. Hum Vaccin Immunother. 2017 Sep. 19: 0.
10. Robert L CTLA4 blockade broadens the peripheral T-cell receptor repertoire. Clin Cancer Res. 2014 May 1;20(9):2424-32. doi: 10.1158/1078-0432.CCR-13-2648. Epub 2014 Feb. 28.
11. Johnson R m et al. Functional Expression of Programmed Death-Ligand 1 (B7-H1) by Immune Cells and Tumor Cells. Front Immunol. 2017 Aug. 10; 8:961
12. Lutz R et al. Immunotherapy converts nonimmunogenic pancreatic tumors into immunogenic foci of immuneregulation. Cancer Immunol Res. 2014 July; 2(7):616-31.
13. Ott P A, et al. An immunogenic personal neoantigen vaccine for patients with melanoma. Nature. 2017 Jul. 13; 547(7662):217-221.
14. Abbas A K, et al. Cellular and Mollecular Immunology Ninth Edition 2018 Elsevier

What is claimed is:

1. A composition comprising:
a) at least one immune checkpoint antagonist; and
b) at least one antigen.

2. The composition of claim 1, wherein the composition comprises at least one nucleic acid encoding the at least one immune checkpoint antagonist.

3. The composition of claim 2, wherein the immune checkpoint antagonist is a polypeptide that binds an immune checkpoint associated protein.

4. The composition of claim 3, wherein the polypeptide is an antibody.

5. The composition of claim 3, wherein the immune checkpoint associated protein is a ligand selected from the group consisting of PD-L1, PD-L2, CD80, CD86, ICOS Ligand, B7-H3, B7-H4, 4-1BBL, HVEM, OX40L, CD70, CD40L, Galectin-9, Adenosine, GITRL, IDO TDO, CEACAM1, VISTA, CD47, CD155, CD48, HHLA2, BTN2A1, BTN2A2, BTN3A1, BTNL2, BTNL3, BTNL9, A2Ar, CD30, CD244, CSFIR, CXCR4-CXCL12, ICOSL, IgSF, ILTs, LIK, MICA/MICB, Neuropilin,NKG2A, phosphatidylserine, Siglec3, TGF-B, TLIA, TNFRSF25, 4.1BBL, A2Ar, BTNL2, CD30, CD244, CSFIR, CXCR4-CXCL12,ICOSL, IgSF, ILTs, LIK, MICA/MICB, Neuropilin,NKG2A, phosphatidylserine, Siglec3, TGF-B, and TLIA.

6. The composition of claim 3, wherein the immune checkpoint associated protein is a soluble receptor selected from the group consisting of PD-1, CTLA-4, CD28, ICOS, 4-1BB, BTLA, CD160, LIGHT, LAG3, OX40, CD27, CD40, TIM-3, Adenosine A2a receptor, GITR, CEACAM1, SIPR-alpha, DNAM-1, TIGIT, CD96, 2B4, TMIGD2, and DC-SIGN.

7. The composition of claim 1, wherein the immune checkpoint antagonist is selected from the group consisting of a CTLA-4 antagonist, vermurafenib, ipilimumab, dacarbazine, IL-2, temozolomide, imatinib, gefitinib, erlotinib, sunitinib, tyrphostins, a PD-1 agonist/antagonist and telatinib .

8. The composition of claim 1, wherein the immune checkpoint antagonist is selected from the group consisting of an siRNA, microRNA, double stranded RNA, dicer substrate RNA, ribozyme and aptamer, TALENs (transcription activator-like effector nuclease), and ZFNS (zinc finger nuclease).

9. The composition of claim 2, wherein the at least one nucleic acid has been codon optimized to yield a more stable mRNA than one encoding the same protein which has not been so optimized.

10. The composition of claim 1, wherein the immune checkpoint antagonist comprises at least one set of CRISPR-CAS9 encoding nucleic acid sequences capable of impairing or eliminating expression of an immune checkpoint associated protein selected from the group consisting of PD-L1, PD-L2, CD80, CD86, ICOS Ligand, B7-H3, B7-H4, 4-1BBL, HVEM, OX40L,CD70, CD40L, Galectin-9, Adenosine, GITRL, IDO, TDO, CEACAM1, VISTA, CD47, CD155, CD48, HHLA2, BTN2A1, BTN2A2, BTN3A1, BTNL3, BTNL9, PD-1, CTLA-4, CD28, ICOS, 4-1BB, BTLA, CD160, LIGHT, LAG3, OX40, CD27, CD40, TIM-3, Adenosine A2a receptor, GITR, CEACAM1, SIPR-alpha, DNAM-1, TIGIT, CD96, 2B4, TMIGD2, and DC-SIGN.

11. The composition of claim 10, wherein the at least one set of CRISPR-CAS9 encoding nucleic acid sequences encodes at least one guide RNA (gRNA) that targets a gene encoding an immune checkpoint associated protein.

12. The composition of claim 1, wherein the antigen comprises a peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, beta-Catenin, CDK4, Mum-1, p16, TAGE, PSMA, PSCA, CT7, telomerase, 43-9F, 5T4, 791Tgp72, alpha-fetoprotein, beta-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, b-amyloid, CA125, CD40, EGFR, G17DT, GD2/3L, gp100, IMA950, KOC1, Peptidyl arginine deiminase-4, MUC-1, OFA, PANVAC, PAP, PSA, PSMA, SL701, SSX-2, TTK, TACAS, URLC10, vEGFR, and WT-1.

13. The composition of claim 1 comprising a nucleotide sequence that encodes the antigen.

14. The composition of claim 1, wherein the antigen is a self-antigen.

15. The composition of claim 1, wherein the antigen is a foreign antigen.

16. The composition of claim 1, wherein the antigen is a tumor associated antigen.

17. The composition of claim 1, wherein the antigen is a cancer-testes associated antigen.

18. The composition of claim 1, wherein the antigen comprises at least one neoantigen polypeptide that is mutated relative to wild type.

19. The composition of claim 18, wherein the at least one mutant polypeptide binds to MHC-Class I or MHC-Class II molecule.

20. The composition of claim 18, wherein the mutation relative to wild type in the neoantigen polypeptide has been identified by sequencing a portion of a tumor cell's genome and comparing that tumor sequence to a corresponding portion of a healthy donor cell's genome.

21. The composition of claim 20, wherein the neoantigen is present across certain patient populations and tumor types.

22. The composition of claim 20, wherein the neoantigen is encoded by an oncogene.

23. The composition of claim 20, wherein the antigen is recognized by a T Cell Receptor (TCR).

24. The composition of claim 20, wherein the sequencing is done by next generation sequencing (NGS).

25. The composition of claim 1, wherein the composition comprises at least one nucleic acid encoding the at least one antigen.

26. The composition of claim 1, wherein the composition comprises at least one nucleic acid encoding the at least one immune checkpoint antagonist and at least one nucleic acid encoding the at least one antigen.

27. The composition of claim 26, wherein the at least one nucleic acid encoding the at least one immune checkpoint antagonist and the at least one nucleic acid encoding the at least one antigen are the same nucleic acid molecule.

28. The composition of claim 26, wherein the at least one nucleic acid encoding the at least one immune checkpoint antagonist and the at least one nucleic acid encoding the at least one immune checkpoint agonist are different molecules.

29. The composition of claim 25, wherein the immune checkpoint antagonist is an antibody.

30. The composition of claim 29, wherein the antibody is selected from the group consisting of a CTLA-4 antagonist, avelumab, pembrolizumab, ipilimumab, durvalumab, atezolizumab, nivolumab, and a PD-1 agonist/antagonist.

31. The composition of claim 25, wherein the at least one nucleic acid is present in a vector.

32. The composition of claim 31, wherein the vector is a lytic vector.

33. The composition of claim 31, wherein the vector is a nanoparticle coated with the antigen epitopes.

34. The composition of claim 27, wherein the same nucleic acid molecule is a single plasmid.

35. The composition of claim 28, wherein the different nucleic acid molecules comprises at least two plasmids.

36. The composition of claim 1 further comprising an adjuvant.

37. The composition of claim 29, wherein the adjuvant is selected from the group consisting of montanide ISA-51, QS-21, tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), incomplete Freunds adjuvant, complete Freunds adjuvant, mineral gels, aluminum hydroxide (Alum), lysolecithin, pluronic polyols, polyanions, peptides, oil emulsions, dinitrophenol, and diphtheria toxin (DT).

38. The composition of claim 1 comprising cells that have been manipulated in vitro to insert the at least one immune checkpoint antagonist and the at least one antigen.

39. The composition of claim 38, wherein the cells are autologous cells.

40. The composition of claim 38, wherein the cells are heterologous cells.

41. The composition of claim 36, wherein the cells are antigen presenting cells (APCs.)

42. A method of treating a disease comprising treating a patient in need thereof with a composition of claim 1.

43. The method of claim 42, wherein the disease is a proliferative disorder.

44. The method of claim 43, wherein the proliferative disorder is cancer.

45. The method of claim 44, wherein the treatment results in at least or about a 10 to 20 percent reduction in cancer tumor size relative to standard treatment without the composition.

46. The method of claim 42, wherein the disease is an infectious disease.

47. The method of claim 42, wherein the disease is neurodegenerative disease.

48. The method of claim 47, wherein the neurodegenerative disease is Alzheimer's disease.

49. The method of claim 42, wherein the disease relates to organ transplant rejection.

50. The method of claim 42, wherein the disease is an autoimmune disease.

51. The method of claim 50, wherein the autoimmune disease is selected from the group consisting of systemic lupus erythematosus, Sjogren's syndrome, SLE, Mixed connective tissue disease, Primary billary cirrhosis, coeliac disease, rheumatoid arthritis, myasthenia Gravis, scleromyositis, Graves disease, Hashimoto's thyroiditis, paraneoplastic cerebellar degeneration, limbic encephalitis, encephalomyelitis, choreathetosis, subacute sensory neuronopathy, stiff person syndrome, diabetes mellitus type 1, optic neuropathy, chorea, and Devics syndrome.

52. A method of preventing a disease comprising treating a patient in need thereof with a composition of claim 1.

53. The method of claim 52, wherein the disease is a proliferative disorder.

54. The method of claim 53, wherein the proliferative disorder is cancer.

55. The method of claim 54, wherein the treatment results in at least or about a 10 to 20 percent reduction in cancer tumor size relative to standard treatment without the composition.

56. The method of claim 52, wherein the disease is an infectious disease.

57. The method of claim 52, wherein the disease is neurodegenerative disease.

58. The method of claim 57, wherein the neurodegenerative disease is Alzheimer's disease.

59. The method of claim 52, wherein the disease relates to organ transplant rejection.

60. The method of claim 52, wherein the disease is an autoimmune disease.

61. The method of claim 60, wherein the autoimmune disease is selected from the group consisting of systemic lupus erythematosus, Sjogren's syndrome, SLE, Mixed connective tissue disease, Primary billary cirrhosis, coeliac disease, rheumatoid arthritis, myasthenia Gravis, scleromyositis, Graves disease, Hashimoto's thyroiditis, paraneoplastic cerebellar degeneration, limbic encephalitis, encephalomyelitis, choreathetosis, subacute sensory neuronopathy, stiff person syndrome, diabetes mellitus type 1, optic neuropathy, chorea, and Devics syndrome.

62. A method of modulating the immune system of a patient in need thereof comprising administering to said patient a composition comprising a neoantigen and an immune checkpoint antagonist in an amount sufficient to elicit an immune response to the neoantigen epitope.

63. The method of claim 62, wherein the neoantigen is selected from the group consisting of a peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Horn/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, beta-Catenin, CDK4, Mum-1, p16, TAGE, PSMA, PSCA, CT7, telomerase, 43-9F, 5T4, 791Tgp72, alpha-fetoprotein, beta-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, b-amyloid, CA125, CD40, EGFR, G17DT, GD2/3L, gp100, IMA950, KOC1, Peptidyl arginine deiminase-4, MUC-1, OFA, PANVAC, PAP, PSA, PSMA, SL701, SSX-2, TTK, TACAS, URLC10, vEGFR, WT-1, BRAF, Pseudomona Exotoxin A, or Diphetheria toxin.

64. The method of claim 62, wherein the antagonist of the checkpoint-associated proteins downregulates the immune system of the patient in response to the neoantigen.

65. A method of modulating the immune system of a patient in need thereof comprising administering to said patient a composition comprising an autoantigen and an immune checkpoint overexpression in an amount sufficient to elicit an immune supression to the autoantigen.

66. The method of claim 65, wherein the autoantigen is selected from the group consisting of histidine-tRNA ligase, Ribonucleoproteins, snRNP core proteins, type I topoisomerase, histones, nucleoporin62, Sp100 nuclear antigen, nucleoporin 210Kda, actin, cyclic citrullinated peptide, thrombin, exosome complex proteins, nicotine acetylcholine receptor, muscle specific kinase, voltage-gated calcium channel, thyroid peroxidase, thyroglobulin, TSH receptor, Neuronal nuclear proteins, glutamate receptor, amphiphysin, glutamate decarboxylase, collapsing response mediator protein 5, N-methyl-D-aspartate receptor, aquaporin, and individual specific autoantibody (ISA).

67. The method of claim 65, wherein the composition further comprising at least one immunosuppressive cytokine.

68. The method of claim 67, wherein the at least one immunosuppressive cytokine is selected from the group consisting of IL-1Ra, IL-4, IL-10, IL-11, IL-13, TGF-b, IL-33, IL-35, and IL-37.

69. A method of generating an animal autoimmune diseases model comprising administering to an animal the composition of claim 1 comprising an autoantigen in order to modulate the immune system of the animal against said autoantige.

70. A method of generating animal autoimmune diseases model comprising administering to an animal the composition of claim 1 comprising an antigen in order to modulate the immune system of the animal against said antigen.
